SEQUENCE LISTING (34 sequences)

SEQ ID NO: 1 - DNA, 309 bp, Vibrio cholerae
  source 1..309: mol_type = other DNA
accccacaaaacatcactgacttgtgtgctgagtaccacaacacccaaatccacaccctcaatgacaagatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaac

SEQ ID NO: 2 - protein, 103 aa, Vibrio cholerae
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 3 - DNA, 327 bp, synthetic construct
  misc_feature 1..327: note = modified cholera toxin B subunit variant polypeptide
  source 1..327: mol_type = other DNA
accccacaaagcatcactgacttgtgtgctgagtaccacaacacccaaatccacaccctcaatgacaagatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaactccgagaaggatgaactc

SEQ ID NO: 4 - protein, 109 aa, synthetic construct
  REGION 1..109: note = modified cholera toxin B subunit variant polypeptide
TPQSITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 5 - DNA, 327 bp, synthetic construct
  misc_feature 1..327: note = modified cholera toxin B subunit variant polypeptide
  source 1..327: mol_type = other DNA
accccacaaaacatcactgacttgtgtgctgagtaccacaacacccaaatccacaccctcaatgacaagatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaactccgagaaggatgaactc

SEQ ID NO: 6 - protein, 109 aa, synthetic construct
  REGION 1..109: note = modified cholera toxin B subunit variant polypeptide
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 7 - DNA, 327 bp, synthetic construct
  misc_feature 1..327: note = modified cholera toxin B subunit variant polypeptide
  source 1..327: mol_type = other DNA
accccacaaaacatcactgacttgtgtgctgagtaccacaacacccaaatccacaccctcaatgacaagatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaacgttactaaggatgaactc

SEQ ID NO: 8 - protein, 109 aa, synthetic construct
  REGION 1..109: note = modified cholera toxin B subunit variant polypeptide
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANVTKDEL

SEQ ID NO: 9 - DNA, 327 bp, synthetic construct
  misc_feature 1..327: note = modified cholera toxin B subunit variant polypeptide
  source 1..327: mol_type = other DNA
accccacaaaacatcactgacttgtgtgctgagtaccacaacacccaaatccacaccctcaatgacactatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaactccgagaaggatgaactc

SEQ ID NO: 10 - protein, 109 aa, synthetic construct
  REGION 1..109: note = modified cholera toxin B subunit variant polypeptide
TPQNITDLCAEYHNTQIHTLNDTIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 11 - DNA, 327 bp, synthetic construct
  misc_feature 1..327: note = modified cholera toxin B subunit variant polypeptide
  source 1..327: mol_type = other DNA
accccacaaaacatcactgacttgtgtgctgagtaccacaacacccaaatccacaccctcaatgacactatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaacgttactaaggatgaactc

SEQ ID NO: 12 - protein, 109 aa, synthetic construct
  REGION 1..109: note = modified cholera toxin B subunit variant polypeptide
TPQNITDLCAEYHNTQIHTLNDTIFSYTESLAGKREMAIITFKNGATFQVEVPGSQHIDSQKKAIERMKDTLRIAYLTEAKVEKLCVWNNKTPHAIAAISMANVTKDEL

SEQ ID NO: 13 - DNA, 345 bp, synthetic construct
  misc_feature 1..345: note = modified cholera toxin B subunit variant polypeptide
  source 1..345: mol_type = other DNA
accccacaaaacatcactgacttgtgtgctgagtaccacaacacccaaatccacaccctcaatgacactatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaacgttactggtggtggaggatccgagaaggatgaactc

SEQ ID NO: 14 - protein, 115 aa, synthetic construct
  REGION 1..115: note = modified cholera toxin B subunit variant polypeptide
TPQNITDLCAEYHNTQIHTLNDTIFSYTESLAGKREMAIITFKNGATFQVEVPGSQHIDSQKKAIERMKDTLRIAYLTEAKVEKLCVWNNKTPHAIAAISMANVTGGGGSEKDEL

SEQ ID NO: 15 - DNA, 393 bp, synthetic construct
  misc_feature 1..393: note = modified cholera toxin B subunit variant polypeptide
  source 1..393: mol_type = other DNA
atggctatcaagctcaagtttggagtgttcttcactgtgctccttagctctgcctatgcacatggcaccccacaaaacatcactgacttgtgtgctgagtaccacaacacccaaatccacaccctcaatgacaagatctttagctacaccgagagccttgctggcaagagggagatggctatcatcaccttcaagaatggtgctaccttccaagtggaggtgcctggaagccaacacattgatagccaaaagaaggccattgagaggatgaaggacacacttaggatagcttacctcactgaggctaaggtggagaagctttgtgtgtggaacaacaagaccccccatgctattgctgccatcagcatggccaactccgagaaggatgaactc

SEQ ID NO: 16 - protein, 131 aa, synthetic construct
  REGION 1..131: note = modified cholera toxin B subunit variant polypeptide
MAIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 17 - DNA, 78 bp, Oryza sativa
  source 1..78: mol_type = other DNA
atggggaagcaaatggccgccctgtgtggctttctcctcgtggcgttgctctggctcacgcccgacgtcgcgcatggt

SEQ ID NO: 18 - protein, 26 aa, Oryza sativa
MGKQMAALCGFLLVALLWLTPDVAHG

SEQ ID NO: 19 - DNA, 81 bp, Nicotiana plumbaginifolia
  source 1..81: mol_type = other DNA
atggctactcaacgaagggcaaaccctagctctctccatctaattactgtattctctctgctcgtcgctgtcgtctcaggt

SEQ ID NO: 20 - protein, 27 aa, Nicotiana plumbaginifolia
MATQRRANPSSLHLITVFSLLVAVVSG

SEQ ID NO: 21 - DNA, 78 bp, Malus domestica
  source 1..78: mol_type = other DNA
atggcattgaagacacagttgttgtggtcattcgtggttgtgttcgttgtgtccttcagtacaacttcatgctcaggt

SEQ ID NO: 22 - protein, 26 aa, Malus domestica
MALKTQLLWSFVVVFVVSFSTTSCSG

SEQ ID NO: 23 - DNA, 72 bp, Hordeum vulgare
  source 1..72: mol_type = other DNA
atggcgaacaaacacttgtccctctccctcttcctcgtcctccttggcctgtcggccagcttggcctcaggt

SEQ ID NO: 24 - protein, 24 aa, Hordeum vulgare
MANKHLSLSLFLVLLGLSASLASG

SEQ ID NO: 25 - protein, 109 aa, synthetic construct
  REGION 1..109: note = modified cholera toxin B subunit variant polypeptide
TPQNITDLCAEYHNTQIHTLNDKIFCYTESLAGKREMAIITFKNGATFQVEVPGSQHIDSQKKAIERMKDTLRIAYLTEAKVEKLCVWNNKTPHAIAAISMCNSEKDEL

SEQ ID NO: 26 - protein, 135 aa, synthetic construct
  REGION 1..135: note = modified cholera toxin B subunit variant polypeptide
MGKQMAALCGFLLVALLWLTPDVAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 27 - protein, 136 aa, synthetic construct
  REGION 1..136: note = modified cholera toxin B subunit variant polypeptide
MATQRRANPSSLHLITVFSLLVAVVSGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 28 - protein, 135 aa, synthetic construct
  REGION 1..135: note = modified cholera toxin B subunit variant polypeptide
MALKTQLLWSFVVVFVVSFSTTSCSGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 29 - protein, 133 aa, synthetic construct
  REGION 1..133: note = modified cholera toxin B subunit variant polypeptide
MANKHLSLSLFLVLLGLSASLASGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

SEQ ID NO: 30 - protein, 6 aa, synthetic construct
  REGION 1..6: note = Endoplasmic Reticulum Retention Signal
SEKDEL

SEQ ID NO: 31 - protein, 4 aa, synthetic construct
  REGION 1..4: note = Endoplasmic Reticulum Retention Signal
KDEL

SEQ ID NO: 32 - protein, 6 aa, synthetic construct
  REGION 1..6: note = Endoplasmic Reticulum Retention Signal
SEHDEL

SEQ ID NO: 33 - protein, 4 aa, synthetic construct
  REGION 1..4: note = Endoplasmic Reticulum Retention Signal
HDEL

SEQ ID NO: 34 - protein, 135 aa, synthetic construct
  REGION 1..135: note = modified cholera toxin B subunit variant polypeptide
MGKQMAALCGFLLVALLWLTPDVAHGTPQSITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSEKDEL

(19) United States
(12) Patent Application Publication
(10) Pub. No.: US 2026/0000732 A1
(43) Pub. Date: Jan. 1, 2026

(54) SPRAY DRIED FORMULATION OF A CHOLERA TOXIN B SUBUNIT VARIANT

(71) Applicant: University of Louisville Research Foundation, Inc., Louisville, KY (US)

(72) Inventors: Krystal Hamorsky, Owensboro, KY (US); Nobuyuki Matoba, Owensboro, KY (US)

(21) Appl. No.: 19/255,186

(22) Filed: Jun. 30, 2025

Related U.S. Application Data
(62) Division of application No. 17/650,386, filed on Feb. 9, 2022, now Pat. No. 12,343,375.
(60) Provisional application No. 63/147,521, filed on Feb. 9, 2021.

(51) Int. Cl. A61K 38/16; A61K 9/00; A61K 9/16

(52) U.S. Cl. CPC A61K 38/164; A61K 9/0053; A61K 9/1623; A61K 9/1694

(57) ABSTRACT

The disclosure provides, in various embodiments, compositions, for example, powders and pharmaceutical compositions, comprising a spray-dried formulation of a cholera toxin B subunit variant and a saccharide excipient. The disclosure also provides, in various embodiments, methods of making said compositions and methods of treating a disease or enhancing wound healing using said compositions. The disclosure further provides, in various embodiments, liquid compositions comprising a cholera toxin B subunit variant and mannitol.

### RELATED APPLICATION

This application is a divisional of U.S. application Ser. No. 17/650,386, filed Feb. 9, 2022, which claims the benefit of U.S. Provisional Application No. 63/147,521, filed on Feb. 9, 2021. The entire teachings of the above applications are incorporated herein by reference.

### GOVERNMENT SUPPORT

This invention was made with government support under DK123712 from the U.S. National Institutes of Health and under U01 HL127518 from the National Institutes of Health. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 5600.1009-002 Sequence Listing.xml; created Jun. 27, 2025, 44,741 Bytes in size.

### BACKGROUND

Ulcerative colitis (UC) is a major form of inflammatory bowel disease (IBD), characterized by chronic and relapsing inflammation of the innermost layer of the colon and rectal mucosa [1,2]. Its etiology remains poorly understood and the onset is associated with a complicated interplay of genetic and environmental factors as well as gut microbiota [3]. The disease often manifests as symptoms including: bloody diarrhea, rectal bleeding, fatigue, and weight loss [4]. However, symptom presentation often varies among patients and may change over time with increasing severity of disease [5]. The Crohn's and Colitis Foundation estimates that approximately 1.6 million Americans suffer from IBD with a total US annual financial burden between USD 14.6 billion and 31.6 billion. UC accounts for 907,000 of these cases with an annual incidence of 12.2 per 100,000 people [5]. Current Food and Drug Administration approved ulcerative colitis (UC) drugs aim to treat existing symptoms, maintain remission, and improve quality of life. Despite multiple treatment options available to UC patients, none of them can cure the disease, and up to a third of those with 30+ years of the disease will require surgical removal of the colon and rectum [5].

There are several classes of drugs used to treat UC [5]. Typically, UC treatment follows a step-up approach in which drug class utilization is dependent upon disease severity and response to prior therapies. The final step of this treatment strategy is surgical intervention [6,7]. Treatment with 5-aminosalicylates (5-ASAs) has long been the mainstay first-line therapy for mild-to-moderate UC [5,8]. This inflammation-blunting class of therapeutics are preferred for early-stage UC because of their generally innocuous side effect profiles, although moderate UC is often unresponsive to these agents [7]. The mild side effect profile is counteracted by the ability of patients to develop tolerance during remission maintenance and require new treatment strategies. Failure to achieve or maintain remission with 5-ASAs is typically followed by treatment with corticosteroids and steroid-sparing immunomodulators. An estimated two-thirds of patients receiving short-term steroid treatment for moderate to severe UC achieve remission. However, the risk of serious adverse effects limits long-term use of these agents [6-9]. Biologics (e.g., anti-TNFα and anti-integrin monoclonal antibodies) have traditionally been the final agents utilized to treat severe UC prior to surgical resection of the colon and rectum, although recent literature suggests the benefit of using biologics in earlier stages [10]. These drugs are effective in remission induction and maintenance in patients following previous treatment failures but are partnered with the serious side effects including severe infection and increased cancer risk [8,9]. Of particular note, fewer than half of patients treated with biologics are able to achieve mucosal healing, an endoscopic marker found to be highly predictive of sustained clinical remission, better quality of life, and decreased risk for colitis-associated colorectal cancer [11,12]. Further, biologics are typically more expensive than other therapeutic agents [13].

### SUMMARY

There is an unmet need in ulcerative colitis (UC) therapy: agents that can directly restore the damaged epithelial barrier and facilitate mucosal healing without suppressing immune function.

In one aspect, the disclosure provides a powder comprising a spray-dried formulation of a cholera toxin B subunit variant and a saccharide excipient.

In another aspect, the disclosure provides a pharmaceutical composition for oral administration, comprising a cholera toxin B subunit variant and a saccharide excipient, wherein the pharmaceutical composition is spray-dried.

In another aspect, the disclosure provides a method of treating a disease, the method comprises administering to a subject in need thereof an effective amount of a pharmaceutical composition comprising a cholera toxin B subunit variant and a saccharide excipient, wherein the pharmaceutical composition is spray-dried.

In another aspect, the disclosure provides a method of enhancing wound healing, the method comprises administering to a subject in need thereof an effective amount of a pharmaceutical composition comprising a cholera toxin B subunit variant and a saccharide excipient, wherein the pharmaceutical composition is spray-dried.

In another aspect, the disclosure provides a method of producing an oral formulation of a cholera toxin B subunit variant, comprising:
a) mixing a phosphate-buffered saline solution of the cholera toxin B subunit variant with a saccharide excipient; and
b) spray drying the solution in an air stream.

In another aspect, the disclosure provides a liquid composition, wherein the liquid composition comprises a cholera toxin B subunit variant and mannitol.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1H depict stability assessment of spray dried EPT. The production of EPT standard is described in the methods section. The stability of dried EPT post-drying and after 9 months was assessed by GM1-capture KDEL-detection ELISA and SEC-HPLC. A representative binding curve of spray dried EPT is shown for one day post-drying (FIGS. 1A and 1E), and after 9 months stored at 23° C. in a desiccator (FIGS. 1B and 1F), compared to an EPT standard. SEC-HPLC chromatogram of non-dried EPT (FIGS. 1C and 1G) and EPT one day post-drying and dried EPT (green) after 9 months stored at 23° C. in a desiccator (FIGS. 1D and 1H). After 9 months dried EPT contained 91.8% pentamer and 8.2% monomer (* represents a line drop as the two peaks are not completely resolved). The resolution value of the two peaks is 1.6 (determined by OpenLab CDS 2.1 software, Agilent Technologies, Santa Clara, CA, USA).

FIG. 2 shows that EPT released from enteric-coated capsules is pH-dependent. Release of EPT was measured by CTB sandwich ELISA. EPT releases from capsules only after submersion at pH 6.8. Percent of EPT release from capsules after 2 h submersion in 0.1 N HCl, and at t=5, 10, 15, 30, 60, 90, 120, and 180 min after pH shift to 6.8. n=5. Percent release was determined by extrapolation of calculated EPT concentrations using CTB sandwich ELISA compared to a known fixed mass of 5 μg EPT per capsule.

FIGS. 3A-3B. Enteric-coated EPT capsules mitigate acute DSS colitis in mice. (FIG. 3A) Study design. (FIG. 3B) Mice were dosed with an EPT capsule (n=10), pre-dissolved EPT powder following gastric acid neutralization (EPT powder solution; n=9), or capsule vehicle control (n=10) on day 7 following DSS exposure. DAI scores were determined on day 14 as a combined measure of body weight recovery, stool consistency, and blood in stool; data are shown as mean±SEM. *p<0.05, ***p<0.001, one-way ANOVA with Bonferroni's multiple comparisons test.

FIGS. 4A-4B. Treatment with encapsulated EPT mitigates acute colitis in mice. Encapsulated EPT (DSS+Capsule) treatment protected mice from histological damage similarly to treatment with EPT powder solution following gastric acid neutralization (DSS+EPT powder solution). (FIG. 4A) Representative 4× (left) and 20× (right) photomicrographs of H&E-stained distal colon tissues from each treatment group. (FIG. 4B) Histological damage scores of each treatment group in the DSS acute colitis study. *p<0.05, **p<0.01; one-way repeated measures ANOVA with Bonferroni's multiple comparisons test.

FIGS. 5A-5C. Evidence for EPT degradation at pH 1.0, These results demonstrate the need for a pH-dependent oral formulation of EPT. FIG. 5A: GM1-CTB ELISA. STD EPT in pentamer has a much higher EC50 to GM1 than acid-exposed EPT which contains EPT in monomer form. FIG. 5B: CTB sandwich ELISA. STD EPT in pentamer form and acid exposed EPT monomer form can be detected in a similar manner demonstrating capacity to detect both GM1-binding pentamer and disassembled CTB molecular species. FIG. 5C: Size Exclusion (SEC)-HPLC. STD EPT elutes in pentamer form at a 17.1-minute retention time whereas acid exposed EPT elutes in monomer form at 19.3 minutes, whereby demonstrating acid dissociates pentamer EPT into monomer EPT.

### DETAILED DESCRIPTION

A description of example embodiments follows.

The subject matter disclosed herein is based, in part, on the disco very of Epicertin (EPT), a dry formulated plant made cholera toxin B subunit CTB^{KDEL}, as a novel, safe, effective and patient accepted treatment for mucosal inflammatory disorders including UC and mucositis.

Non-limiting benefits of dry-formulated Epicertin include improved patient adherence due to ease of administration and mitigate bad taste or smell; effortless formulation for targeted delivery; avoiding cold chain requirements; and improved product shelf life. Longer processing times, smaller dryer size, batch mode of production, and chill injury are considerable disadvantages of freezing drying process. In addition, the capital and operational costs of the spray drying process are 1/9 and ⅙, respectively, compared with those of freeze drying industrial technique.

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As would be recognized by those of ordinary skill in the art, cholera toxin is an oligomeric protein complex, which is secreted by the bacterium Vibrio cholerae and is thought to be responsible for the enteric symptoms characteristic of a cholera infection. The cholera toxin itself is generally composed of six protein subunits, namely a single copy of the A subunit, which is thought to be the toxic portion of the molecule responsible for its enzymatic action; and five copies of the B subunit, which form a pentameric ring and are thought to comprise the non-toxic portions of the molecule responsible for binding to receptors, such as the GM1 ganglioside receptor, which contains a glycosphingolipid (e.g., a ceramide and oligosaccharide) with one sialic acid and which is attached to the surface of a host cell. As such, the term “cholera toxin B subunit” is used herein to refer to a single B subunit of the cholera toxin as well as to B subunits of the cholera toxin in the form of multimers (e.g., in a pentameric form). Exemplary nucleic acid and amino acid sequence of a native cholera toxin B subunit polypeptide from wild-type Vibrio cholerae are provided herein in SEQ ID NOs: 1 and 2 (see the section entitled “Sequences”).

As used herein, the term “variant” refers to a polypeptide comprising an amino acid sequence that has at least about 70% sequence identity to a reference sequence, i.e., a wild type cholera toxin B subunit.

The term “polypeptide” “peptide” or “protein” denotes a polymer of at least two amino acids covalently linked by an amide bond, regardless of length or post-translational modification (e.g., glycosylation or phosphorylation). A protein, peptide or polypeptide can comprise any suitable L-and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a protein, peptide or polypeptide can also be derivatized (e.g., alkylated) or labeled (e.g., with a detectable label, such as a fluorogen or a hapten) using methods known in the art. A protein, peptide or polypeptide can comprise one or more modifications (e.g., amino acid linkers, acylation, acetylation, amidation, methylation, terminal modifiers (e.g., cyclizing modifications), N-methyl-α-amino group substitution), if desired. In addition, a protein, peptide or polypeptide can be an analog of a known and/or naturally-occurring peptide, for example, a peptide analog having conservative amino acid residue substitution(s).

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., Basic local alignment search tool. J Mol Biol. 215 (3): 403-10 (1990)).

The term “conservative amino acid substitution(s)” or “conservative substitution(s)” refers to an amino acid substitution having a value of 0 or greater in BLOSUM62.

The term “highly conservative amino acid substitution(s)” or “highly conservative substitution(s)” refers to an amino acid substitution having a value of at least 1 (e.g., at least 2) in BLOSUM62.

As used herein, the term “pharmaceutically acceptable excipient” includes, e.g., suitable solvents, dispersion media, coatings, antibacterial agents and antifungal agents, isotonic agents, and absorption delaying agents. The use of such media and agents for pharmaceutically active substances is known in the art.

As used herein, the terms “treatment” or “treating” relate to any treatment of a disease of a subject, including, but not limited to, prophylactic treatment and therapeutic treatment. As such, the terms treatment or treating include, but are not limited to: preventing a disease or the development of a disease; inhibiting the progression of a disease; arresting or preventing the development of a disease; reducing the severity of a disease; ameliorating or relieving symptoms associated with a disease; and causing a regression of the disease or one or more of the symptoms associated with the disease.

The term “cancer” refers to all types of cancer or neoplasm or malignant tumors found in a subject, including leukemias, lymphomas, myelomas, carcinomas, melanomas, teratomas, and sarcomas. Examples of cancers include cancer of the liver, pancreas, esophagus, brain, bladder, breast, central nervous system (e.g., spine), cervix, colon, rectum, head and neck, kidney, lung, ovary, prostate, sarcoma, stomach, uterus, leukemias, lymphomas, myelomas, and melanomas.

The term “inflammatory disorder” includes diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some inflammatory disorders fall within one or more categories.

Non-limiting examples of inflammatory disorders include atherosclerosis, arthritis, asthma, autoimmune uveitis, adoptive immune response, dermatitis, multiple sclerosis, diabetic complications, osteoporosis, Alzheimer's disease, cerebral malaria, hemorrhagic fever, autoimmune disorders, and inflammatory bowel disease. In some embodiments, the term “inflammatory disorder” is further inclusive of inflammation-promoted cancers, such that the term “inflammatory disorder” can be used to refer to cancers caused or promoted by inflammation, such as colon cancer.

The term “colitis” refers to an inflammation of the colon which may be acute or chronic.

The term “subject” refers to a mammal (e.g., human, dog, cat, horse, cow, mouse, rat). Preferably, the subject is a human (e.g., a human who has, or is at risk for developing cancer). A “subject in need thereof” refers to a subject who has, or is at risk for developing, cancer. A skilled medical professional (e.g., physician) can readily determine whether a subject (e.g., a patient) has, or is at risk for developing, cancer.

“A therapeutically effective amount” is an amount effective, at dosages and for periods of time necessary, to achieve a desired therapeutic result (e.g., treatment, healing, inhibition or amelioration of physiological response or condition, etc.). Example indicators of a “therapeutically effect amount” include, e.g., improved well-being of the patient, reduction of tumor burden, arrested or slowed growth of a tumor, or absence of metastasis of cancer cells, or a combination thereof.

“In combination with” means that two or more therapeutics can be administered to a subject together in a mixture or composition, concurrently as single agents, or sequentially as single agents in any order.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the invention, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

### Spray-Dried Powders

In one aspect, the disclosure provides a powder comprising a spray-dried formulation of a cholera toxin B subunit variant and a saccharide excipient.

### Cholera Toxin B Subunit Variants

In some embodiments, the cholera toxin B subunit variant has at least about 80% sequence identity to a wild-type cholera toxin B subunit (e.g., SEQ ID NO:2), for example, having at least about: 85%, 90%, 91%, 92%, 93%, 94%, 95%, 96%, 97%, 98% or 99% sequence identity or having about: 80-99%, 85-99%, 90-99%, 91-99%, 92-99%, 93-99%, 94-99%, 95-99%, 96-99%, 97-99% or 98-99% sequence identity.

In particular embodiments, the cholera toxin B subunit variant comprises an endoplasmic reticulum (ER) retention sequence attached to its C-terminus. In some embodiments, the C-terminal ER retention sequence comprises SEKDEL (SEQ ID NO:30), KDEL (SEQ ID NO:31), SEHDEL (SEQ ID NO:32) or HDEL (SEQ ID NO:33).

In certain embodiments, the cholera toxin B subunit variant comprises an amino acid sequence that has at least about 80% sequence identity to at least one sequence set forth in SEQ ID NOs: 4, 6, 8, 10, 12, 14, 16, 25-29 and 34 (see the section entitled “Sequences”), for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:4, 6, 8, 10, 12, 14, 16, 25-29 and 34.

In particular embodiments, the cholera toxin B subunit variant comprises secretory signal peptide (e.g., a N-terminal secretory signal peptide). In some embodiments, the secretory signal peptide is a rice alpha-amylase secretory signal peptide, a Nicotiana plumbagenifolia calreticulin secretory signal peptide, an apple pectinase secretory signal peptide, or a barley alpha-amylase secretory signal peptide. In some embodiments, the secretory signal peptide sequence comprises SEQ ID NO:18, 20, 22 or 24.

In certain embodiments, the cholera toxin B subunit variant comprises an amino acid sequence that has at least about 80% sequence identity to at least one sequence set forth in SEQ ID NOs: 16, 26-29 and 34, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO: 16, 26, 27, 28, 29 or 34.

In some embodiments, the cholera toxin B subunit variant comprises an amino acid sequence that has at least about 80% sequence identity to SEQ ID NO:4 and/or SEQ ID NO:34, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:4 or SEQ ID NO:34.

In some embodiments, the cholera toxin B subunit variant comprises an amino acid sequence that has at least about 80% sequence identity to SEQ ID NO:4, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:4.

In certain embodiments, the cholera toxin B subunit variant comprises an amino acid sequence that has at least about 80% sequence identity to SEQ ID NO:6, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:6.

In some embodiments, the cholera toxin B subunit variant comprises an amino acid sequence that has at least about 80% sequence identity to SEQ ID NO:8, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:8.

In certain embodiments, the cholera toxin B subunit variant comprises an amino acid sequence that has at least about 80% sequence identity to SEQ ID NO:10, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:10.

In some embodiments, the cholera toxin B subunit variant comprises an amino acid sequence that has at least about 80% sequence identity to SEQ ID NO:12, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:12.

In certain embodiments, the cholera toxin B subunit variant comprises an amino acid sequence that has at least about 80% sequence identity to SEQ ID NO:14, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:14.

In some embodiments, the cholera toxin B subunit variant comprises an amino acid sequence that has at least about 80% sequence identity to SEQ ID NO:16, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO: 16.

In certain embodiments, the cholera toxin B subunit variant comprises an amino acid sequence that has at least about 80% sequence identity to SEQ ID NO:25, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:25.

In some embodiments, the cholera toxin B subunit variant comprises an amino acid sequence that has at least about 80% sequence identity to SEQ ID NO:26, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:26.

In certain embodiments, the cholera toxin B subunit variant comprises an amino acid sequence that has at least about 80% sequence identity to SEQ ID NO:27, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:27.

In some embodiments, the cholera toxin B subunit variant comprises an amino acid sequence that has at least about 80% sequence identity to SEQ ID NO:28, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:28.

In certain embodiments, the cholera toxin B subunit variant comprises an amino acid sequence that has at least about 80% sequence identity to SEQ ID NO:29, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:29.

In some embodiments, the cholera toxin B subunit variant comprises an amino acid sequence that has at least about 80% sequence identity to SEQ ID NO:34, for example, having at least about: 85%, 90%, 91%, 92%, 93%, 94%, 95%, 96%, 97%, 98%, 99% or 100% sequence identity or having about: 80-100%, 85-100%, 90-100%, 91-100%, 92-100%, 93-100%, 94-100%, 95-100%, 96-100%, 97-100% or 98-100% sequence identity. In particular embodiments, the cholera toxin B subunit variant comprises an amino acid sequence set forth in SEQ ID NO:34.

In certain embodiments, the cholera toxin B subunit variant comprises at least one amino acid substitution, insertion, or deletion relative to a wild-type cholera toxin B subunit (e.g., SEQ ID NO:2). For example, the cholera toxin B subunit variant comprises at least: 2, 3, 4, 5, 6, 7, 8, 9, 10, 11, 12, 13, 14, 15, 16, 17, 18, 19 or 20 amino acid substitutions relative to a wild-type cholera toxin B subunit. In some embodiments, the cholera toxin B subunit variant comprises about: 1-20, 1-18, 2-18, 2-15, 3-15, 3-12, 4-12, 4-10, 5-10, 5-8 or 6-8 amino acid substitutions, relative to the reference sequence. In some embodiments, the amino acid substitutions include at least one conservative substitution. In certain embodiments, the amino acid substitutions include at least one highly conservative substitution.

In particular embodiments, the cholera toxin B subunit variant comprises an Asn4 to Ser mutation.

In some embodiments, the one or more modification (amino acid substitution, insertion, and/or deletion) facilitates recombinant production of the cholera toxin B subunit variant. In certain embodiments, the cholera toxin B subunit variant has an increased level of expression in a plant cell than the corresponding wild-type cholera toxin B subunit. In particular embodiments, the cholera toxin B subunit variant is substantially immunologically identical to a wild-type cholera toxin B subunit.

In some embodiments, the powder comprises one cholera toxin B subunit variant. In other embodiments, the powder comprises two or more cholera toxin B subunit variants, for example, 2, 3, 4, 5 or more cholera toxin B subunit variants.

### Saccharide Excipients

Non-limiting examples of saccharide excipients suitable for use in the disclosure include monosaccharides, disaccharides, polysaccharides and alditols.

In some embodiments, the saccharide excipient comprises an alditol. Non-limiting examples of alditols include lactitol, maltitol, mannitol, myoinositol, pyranosyl sorbitol, xylitol, xylitol sorbitol (glucitol), and combinations thereof. In particular embodiments, the saccharide excipient comprises mannitol.

In certain embodiments, the saccharide excipient comprises a monosaccharide. Non-limiting examples of monosaccharides include dextrose, fructose, maltose, galactose, glucose, D-mannose, sorbose, and combinations thereof. In particular embodiments, the saccharide excipient comprises dextrose.

In some embodiments, the saccharide excipient comprises a disaccharide. Non-limiting examples of disaccharides include lactose, sucrose, trehalose, cellobiose, and combinations thereof. In particular embodiments, the saccharide excipient comprises lactose, trehalose, or both.

In certain embodiments, the saccharide excipient comprises a polysaccharide. Non-limiting examples of polysaccharides include raffinose, melezitose, maltodextrins, dextrans, starches, and combinations thereof.

In some embodiments, the saccharide excipient is thermoprotective. In certain embodiments, the saccharide excipient comprises mannitol, dextrose, trehalose, lactose, or a combination thereof. In some embodiments, the saccharide excipient comprises mannitol, lactose, or both.

In some embodiments, the saccharide excipient is inert (e.g., has a relatively low hygroscopicity, is relatively less likely to react with active pharmaceutical ingredients, or both).

In some embodiments, the powder comprises one saccharide excipient. In other embodiments, the powder comprises two or more saccharide excipients, for example, 2, 3, 4, 5 or more saccharide excipients.

### Pharmaceutical Compositions

In some embodiments, a powder of the disclosure is formulated into a pharmaceutical composition suitable for administration (e.g., for oral administration) to a subject such as a mammal, e.g., a human patient.

In another aspect, the disclosure provides a pharmaceutical composition for oral administration, comprising a cholera toxin B subunit variant and a saccharide excipient, wherein the pharmaceutical composition is spray-dried.

The cholera toxin B subunit variant can be any one or more of the cholera toxin B subunit variants described herein. In some embodiments, the cholera toxin B subunit variant comprises an amino acid sequence that has at least about 80% sequence identity to SEQ ID NO:4 and/or SEQ ID NO:34. In certain embodiments, the cholera toxin B subunit variant comprises an amino acid sequence set forth in SEQ ID NO:4 or SEQ ID NO:34.

The saccharide excipient can be any one or more of the saccharide excipients described herein. In particular embodiments, the saccharide excipient comprises mannitol.

In certain embodiments, the pharmaceutical composition further comprises one or more pharmaceutically acceptable excipients.

A pharmaceutical composition of the disclosure can be formulated to be compatible with its intended route of administration. For example, oral compositions can include an inert diluent or an edible carrier. They can be enclosed in gelatin capsules or compressed into tablets. The pharmaceutical composition can be in the form of tablets, troches, or capsules. In certain embodiments, the pharmaceutical composition is in the form of a capsule.

In some embodiments, the pharmaceutical composition (e.g., capsule) is enteric-coated.

In certain embodiments, the capsule (e.g., the cap, the body joints, or both) comprises a polymer that degrades at a desired pH. In some embodiments, the desired pH is between about pH 6.7 and about pH 6.9, for example, about pH 6.7, pH 6.8 or pH 6.9. In some embodiments, the capsule comprises a polymer that degrades at about pH 6.8. In particular embodiments, the capsule comprises Eudragit® L100 anionic polymer.

In particular embodiments, the pharmaceutical composition is encapsulated at about pH 1 and gradually released at about pH 6.8.

In certain embodiments, the pharmaceutical composition:
a) comprises no more than about 25% monomer;
b) comprises no more than about 10% moisture; or
c) exhibits about 100±20% solubility, or
a combination thereof.

In some embodiments, the pharmaceutical composition comprises no more than about 25% monomer, for example, no more than about: 20%, 18%, 15%, 12%, 10%, 9%, 8%, 7%, 6%, 5%, 4%, 3.5%, 3%, 2%, 1.5% or 1% monomer. In certain embodiments, the pharmaceutical composition comprises no more than about 10% monomer. In certain embodiments, the pharmaceutical composition comprises less than about 5% monomer. In some embodiments, the pharmaceutical composition comprises less than about 3.5% monomer. In particular embodiments, the pharmaceutical composition comprises less than about 1.5% monomer.

In certain embodiments, the pharmaceutical composition comprises about 1-20% monomer, for example, about: 1-18%, 1-15%, 1-12%, 1-10%, 1-9%, 1-8%, 1-7%, 1-6%, 1-5%, 1-4%, 1-3%, 1-2%, 2-20%, 2-18%, 2-15%, 2-12%, 2-10%, 2-9%, 2-8%, 2-7%, 2-6%, 2-5%, 2-4%, 2-3%, 3-20%, 3-18%, 3-15%, 3-12%, 3-10%, 3-9%, 3-8%, 3-7%, 3-6%, 3-5%, 3-4%, 4-20%, 4-18%, 4-15%, 4-12%, 4-10%, 4-9%, 4-8%, 4-7%, 4-6% or 4-5% monomer. In particular embodiments, the pharmaceutical composition comprises about 1-3.5% monomer.

In certain embodiments, the pharmaceutical composition comprises no more than about 10% moisture, for example, no more than about: 9%, 8%, 7%, 6%, 5%, 4%, 4%, 2% or 1% moisture. In particular embodiments, the pharmaceutical composition comprises about 1-10% moisture, for example, about: 1-9%, 1-8%, 1-7%, 1-6%, 1-5%, 1-4%, 1-3%, 1-2%, 2-10%, 2-9%, 2-8%, 2-7%, 2-6%, 2-5%, 2-4%, 2-3%, 3-10%, 3-9%, 3-8%, 3-7%, 3-6%, 3-5%, 3-4%, 4-10%, 4-9%, 4-8%, 4-7%, 4-6%, 4-5%, 5-10%, 5-9%, 5-8%, 5-7%, 5-6%, 6-10%, 6-9%, 6-8% or 6-7% moisture.

In some embodiments, the pharmaceutical composition exhibits about 100±20% solubility, for example, about: 100±18%, 100±15%, 100±12%, 100±10%, 100±9%, 100±8%, 100±7%, 100±6% or 100±5% solubility. In particular embodiments, the pharmaceutical composition exhibits about 100±10% solubility.

In certain embodiments, the pharmaceutical composition:
a) comprises no more than about 5% monomer;
b) comprises no more than about 10% moisture; or
c) exhibits about 100±10% solubility, or
a combination thereof.

In some embodiments, less than about 10%, for example, less than about: 9%, 8%, 7%, 6%, 5%, 4%, 3%, 2% or 1% or about: 1-10%, 1-5%, 1-3%, 3-10%, 3-5% or 5-10%, of the cholera toxin B subunit variant pentamer in the pharmaceutical composition degrades after a 9-month post-drying storage at about 22-24° C. In certain embodiments, less than about 5% of the cholera toxin B subunit variant pentamer in the pharmaceutical composition degrades after a 9-month post-drying storage at about 23° C. In particular embodiments, less than about 3% of the cholera toxin B subunit variant pentamer in the pharmaceutical composition degrades after a 9-month post-drying storage at about 23° C.

### Methods of Treatment

In another aspect, the disclosure provides a method of treating a disease, the method comprises administering to a subject in need thereof an effective amount of a pharmaceutical composition comprising a cholera toxin B subunit variant and a saccharide excipient, wherein the pharmaceutical composition is spray-dried.

In another aspect, the disclosure provides a method of enhancing wound healing, the method comprises administering to a subject in need thereof an effective amount of a pharmaceutical composition comprising a cholera toxin B subunit variant and a saccharide excipient, wherein the pharmaceutical composition is spray-dried.

The cholera toxin B subunit variant can be any one or more of the cholera toxin B subunit variants described herein. In some embodiments, the cholera toxin B subunit variant comprises an amino acid sequence that has at least about 80% sequence identity to SEQ ID NO:4 and/or SEQ ID NO:34. In certain embodiments, the cholera toxin B subunit variant comprises an amino acid sequence set forth in SEQ ID NO:4 or SEQ ID NO:34.

The saccharide excipient can be any one or more of the saccharide excipients described herein. In particular embodiments, the saccharide excipient comprises mannitol.

In some embodiments, the disease is cancer. In certain embodiments, the cancer is colon cancer. In particular embodiments, the colon cancer is colitis-associated colon cancer.

In some embodiments, the disease is an inflammatory disorder. In particular embodiments, the inflammatory disorder is sepsis, septic shock, colitis, colon cancer or arthritis. In certain embodiments, the inflammatory disorder is an inflammatory bowel disease, a gastrointestinal inflammation and/or injury, a mucosal inflammation, or a combination thereof.

In some embodiments, the inflammatory bowel disease comprises ulcerative colitis, Crohn's disease, or both. In certain embodiments, the inflammatory bowel disease comprises Crohn's disease. In particular embodiments, the inflammatory bowel disease comprises ulcerative colitis. In some embodiments, the inflammatory bowel disease comprises ulcerative colitis and Crohn's disease.

Non-limiting examples of gastrointestinal inflammations and/or injuries include celiac disease, irritable bowel syndrome, radiation-induced colitis, infection-induced colitis, and combinations thereof.

Non-limiting examples of mucosal inflammations and/or injuries include asthma, airway burns, corneal injury, vaginosis, and combinations thereof.

In some embodiments, the disease is a mucosal inflammatory disorder. In certain embodiments, the disease comprises ulcerative colitis, mucositis or both. In some embodiments, the disease comprises ulcerative colitis and mucositis. In particular embodiments, the disease comprises mucositis.

In particular embodiments, the pharmaceutical composition is administered in combination with one or more additional therapeutics. In some embodiments, the pharmaceutical composition and the one or more additional therapeutics are administered together. In other embodiments, the pharmaceutical composition and the one or more additional therapeutics are administered separately.

### Methods of Making

In another aspect, the disclosure provides a method of producing an oral formulation of a cholera toxin B subunit variant, comprising:
a) mixing a phosphate-buffered saline solution of the cholera toxin B subunit variant with a saccharide excipient; and
b) spray drying the solution in an air stream.

The cholera toxin B subunit variant can be any one or more of the cholera toxin B subunit variants described herein. In some embodiments, the cholera toxin B subunit variant comprises an amino acid sequence that has at least about 80% sequence identity to SEQ ID NO:4 and/or SEQ ID NO:34. In certain embodiments, the cholera toxin B subunit variant comprises an amino acid sequence set forth in SEQ ID NO:4 or SEQ ID NO:34.

The saccharide excipient can be any one or more of the saccharide excipients described herein. In some embodiments, the saccharide excipient comprises mannitol. In certain embodiments, the method comprises mixing a phosphate-buffered saline solution of the cholera toxin B subunit variant (e.g., at about 1 mg/mL) with about 100 mM mannitol. In particular embodiments, the method comprises mixing a phosphate-buffered saline solution of the cholera toxin B subunit variant (e.g., at about 1 mg/mL) with about 100 mM mannitol at about pH 7.2.

In some embodiments, spray drying the solution uses:
a) an inlet air temperature of about 116-122° C.;
b) an outlet air temperature of about 64-78° C.; or
c) an inlet to outlet air temperature ratio of about 1.7-2.1, or
a combination thereof.

In particular embodiments, spray drying the solution uses:
a) an inlet air temperature of about 116-122° C.;
b) an outlet air temperature of about 64-67° C.; or
c) an inlet to outlet air temperature ratio of about 1.7-2.1, or
a combination thereof.

In certain embodiments, spray drying the solution uses an inlet air temperature of about 116-122° C., for example, an inlet air temperature of about: 116° C., 117° C., 118° C., 119° C., 120° C., 121° C. or 122° C.

In particular embodiments, spray drying the solution uses an outlet air temperature of about 64-78° C., for example, an outlet air temperature of about: 64° C., 65° C., 66° C., 67° C., 68° C., 69° C., 70° C., 71° C., 72° C., 73° C., 74° C., 75° C., 76° C., 77° C. or 78° C., or an outlet air temperature of about: 64-78° C., 64-77° C., 64-76° C., 64-75° C., 64-74° C., 64-73° C., 64-72° C., 64-71° C., 64-70° C., 64-69° C., 64-68° C. or 64-67° C. In some embodiments, spray drying the solution uses an inlet air temperature of about 64-67° C.

In certain embodiments, spray drying the solution uses an inlet to outlet air temperature ratio of about 1.7-2.1, for example, an inlet to outlet air temperature ratio of about: 1.7, 1.8, 1.9, 2.0 or 2.1 or an inlet to outlet air temperature ratio of about: 1.7-2.0, 1.7-1.9, 1.7-1.8, 1.8-2.1, 1.8-2.0, 1.8-1.9, 1.9-2.1, 1.9-2.0 or 2.0-2.1.

In some embodiments, the Q-flow (drying gas flow rate) is about 34 or 35 g/min. In certain embodiments, the Q-flow is about 34 g/min. In particular embodiments, the Q-flow is about 35 g/min.

In certain embodiments, spray drying the solution uses an aspirator setting of about 80 to 95, for example, about: 80, 85, 90 or 95, or about 80-90, 80-85, 85-95, 85-90 or 90-95.

In certain embodiments, spray drying the solution uses a pump setting of about 20-30, for example, about: 20, 21, 22, 23, 24, 25, 26, 27, 28, 29 or 30, or about 20-28, 20-25, 20-22, 22-30, 22-28, 22-25, 25-30, 25-28 or 28-30.

### Liquid Compositions

In another aspect, the disclosure provides a liquid composition, wherein the liquid composition comprises a cholera toxin B subunit variant and mannitol.

The cholera toxin B subunit variant can be any one or more of the cholera toxin B subunit variants described herein. In some embodiments, the cholera toxin B subunit variant comprises an amino acid sequence that has at least about 80% sequence identity to SEQ ID NO:4 and/or SEQ ID NO:34. In certain embodiments, the cholera toxin B subunit variant comprises an amino acid sequence set forth in SEQ ID NO:4 or SEQ ID NO:34.

In some embodiments, the liquid composition comprises about 100 mM mannitol. In certain embodiments, the liquid composition further comprises phosphate buffered saline (PBS).

In certain embodiments, the liquid composition comprises about 2.7 mM KCl, about 1.5 mM KH₂PO₄, about 136.9 mM NaCl, about 8.9 mM Na₂HPO₄·7H₂O, about 100 mM mannitol, or a combination thereof. In particular embodiments, the liquid composition comprises about 2.7 mM KCl, about 1.5 mM KH₂PO₄, about 136.9 mM NaCl, about 8.9 mM Na₂HPO₄·7H₂O and about 100 mM mannitol.

### EXAMPLES

Mucosal healing is a major treatment goal for UC patients [11,12,14,15]. It is a complex and dynamic process involving multiple cell types including epithelial, stromal and immune cells [14]. Epithelial repair plays a crucial role in mucosal healing by rebuilding the intestinal barrier to inhibit inflammation caused by entry of bacteria into the mucosa. Since inflammation in UC is limited to the innermost layer of the colon and rectal mucosa, epithelial repair may be key to achieving mucosal healing in this subset of IBD patients [5,11]. Thus, an epithelial repair agent may fill a current treatment gap for UC. We previously found that oral administration of a plant-made recombinant variant of cholera toxin B subunit (CTB) facilitates epithelial repair and mucosal healing in dextran sulfate sodium (DSS)-induced acute and chronic colitis mouse models [15,16]. CTB is the nontoxic homopentameric component of the cholera toxin with high binding affinity to GM1 ganglioside on epithelial cells [17]. This variant, CTB^{KDEL}, henceforth designated Epicertin (EPT), has a major modification from the parent molecule; the C-terminal hexapeptide extension containing a KDEL endoplasmic reticulum (ER) retention motif [18,19]. While the alteration did not affect the GM1-binding affinity, molecular stability or oral immunogenicity of the original molecule [18], EPT, but not wild-type CTB, induced mucosal healing in the DSS colitis model. This unique new activity, which stems from EPT's capacity to interact with the KDEL receptor and subsequently activate the inositol-requiring enzyme 1/X-box binding protein 1 arm of an unfolded protein response in colon epithelial cells [20], lends support for the development of EPT as a new class of oral therapeutics for UC.

EPT may be administered to the colon topically or by oral gavage to alleviate DSS induced colitis in mice [20]. Although oral medications are generally preferred by patients and increase patient adherence to treatment regimens [3,21,22], oral administration of EPT solution requires neutralization of stomach acid to prevent degradation of the protein. This is similar to the World Health Organization prequalified oral cholera vaccine, Dukoral™, which is administered in a solution following stomach neutralization with a sodium bicarbonate solution. Considering potential long-term treatment necessary for the management of UC [21], this neutralization step could be disadvantageous as it would likely lower patient adherence and ease of administration. To address this limitation, we describe here the development of a prototype enteric-coated oral formulation of EPT that allows for pH-dependent release of the drug substance in the colon, wherein the protein was spray dried and encapsulated in a gelatin capsule coated with an anionic polymer. Our results provide a foundation for further development of a novel oral biologic to facilitate colon mucosal healing in UC.

### Example 1

### Materials and Methods

### Animals

Eight-week-old C57BL/6J, female mice were obtained from Jackson Laboratories (Bar Harbor, ME). The University of Louisville's Institutional Animal Care and Use Committee approved all animal studies conducted herein.

### EPT Production

EPT was produced in Nicotiana benthamiana using a transient overexpression system and purified to >95% homogeneity with an endotoxin level of <3 endotoxin units per mg as described previously [19,22,23]. EPT was ultrafiltrated/diafiltrated into various buffers (Tables 1A-1B) using 30,000 MWCO centrifugal devices. Phosphate buffered saline (PBS) with 100 mM mannitol was found to be the optimal buffer for spray drying of EPT. EPT at 1 mg/mL in PBS with 100 mM mannitol excipient (pH 7.2) was dehydrated using a Büchi B-290 mini spray drier with an inlet temperature of 125° C. and an outlet temperature maintained between 65° C. and 67° C. The Q-Flow was 35 mm, aspirator was 90% and pump 20%. EPT powder was stored in conical tubes wrapped in parafilm under desiccation at room temperature (20-25° C.) until use. Standard EPT used for the GM1/KDEL ELISA and SEC-HPLC was produced in Nicotiana benthamiana using a transient overexpression system and purified to >95% homogeneity with an endotoxin level of <3 endotoxin units per mg as described previously [19,22,23].

TABLE 1A
Determination of optimal buffer excipient for CTB^{KDEL} (EPT) powder production.

| | % | % | % | % |
|---|---|---|---|---|
| Buffer | Monomer | Moisture | Loss | Solubility |
| PBS | 4.9 | 2.9 | 29.3 | 106 |
| PBS, 20 mM Mannitol | 4.5 | 10.3 | 56.6 | 94 |
| PBS, 100 mM Mannitol | 3.1 | 0 | 60.4 | 97 |
| PBS, 250 mM Mannitol | 9.1 | 7.5 | 80.4 | 99 |
| PBS, 250 mM Mannitol | 6.4 | 4.1 | 68.1 | 87 |
| 30 mM Phosphate, pH 7 | 3.2 | 31.5 | 38.5 | 67 |
| 30 mM Phosphate, 20 mM Mannitol, pH 7 | 1.1 | 14.7 | 79.5 | 81 |
| 30 mM Phosphate, 250 mM Mannitol, pH 7 | 5.8 | 2.3 | 28.9 | 97 |
| 30 mM Phosphate, 250 mM Mannitol, pH 7 | 5.8 | 3.9 | 43.5 | 96 |
| 30 mM Phosphate, 250 mM Mannitol, pH 7 | 6.7 | 5.4 | 33.2 | 91 |
| 30 mM Phosphate, pH 7.5 | 6.1 | 20 | 73.1 | 79 |
| 30 mM Phosphate, 20 mM Mannitol, pH 7.5 | 2.4 | 21.3 | 63.6 | 82 |
| 30 mM Phosphate, 250 mM Mannitol, pH 7.5 | 5.2 | 1 | 28.5 | 88 |
| 30 mM Phosphate, 250 mM Mannitol, pH 7.5 | 7.0 | 2.1 | 34.4 | 92 |
| 30 mM Phosphate, 250 mM Mannitol, pH 7.5 | 7.3 | 3.1 | 50.2 | 64 |
| 88 mM Phosphate, 20 mM Mannitol, pH 7 | 2.1 | 18.4 | 43.7 | 96 |
| TARGET | <5% | <10% | <40% | 100 ± 10 |

TABLE 1B
Determination of optimal buffer excipient for CTB^{KDEL} (EPT) powder production.

| | % | % | % | % |
|---|---|---|---|---|
| Buffer | Monomer | Moisture | Loss | Solubility |
| PBS | 12.3 | 9.7 | 36.2 | 97 |
| PBS | 5.9 | 4.5 | 34.5 | 101 |
| PBS | 3.2 | 32.3 | 29.3 | 101 |
| PBS | 8.0 | 3.7 | 36.2 | 99 |
| PBS | 4.9 | 2.9 | 29.3 | 106 |
| PBS, 20 mM Mannitol | 4.5 | 10.3 | 56.6 | 94 |
| PBS, 100 mM Mannitol | 4.4 | 3.9 | 60.4 | 101 |
| PBS, 100 mM Mannitol | 3.1 | 0 | 60.4 | 97 |
| PBS, 250 mM Mannitol | 9.1 | 7.5 | 80.4 | 99 |
| PBS, 250 mM Mannitol | 6.4 | 4.1 | 68.1 | 87 |
| 30 mM Phosphate, pH 7 | 3.2 | 31.5 | 38.5 | 67 |
| 30 mM Phosphate, 20 mM Mannitol, pH 7 | 1.1 | 14.7 | 79.5 | 81 |
| 30 mM Phosphate, 250 mM Mannitol, pH 7 | 5.8 | 2.3 | 28.9 | 97 |
| 30 mM Phosphate, 250 mM Mannitol, pH 7 | 5.8 | 3.9 | 43.5 | 96 |
| 30 mM Phosphate, 250 mM Mannitol, pH 7 | 6.7 | 5.4 | 33.2 | 91 |
| 30 mM Phosphate, pH 7.5 | 6.1 | 20 | 73.1 | 79 |
| 30 mM Phosphate, 20 mM Mannitol, pH 7.5 | 2.4 | 21.3 | 63.6 | 82 |
| 30 mM Phosphate, 250 mM Mannitol, pH 7.5 | 5.2 | 1 | 28.5 | 88 |
| 30 mM Phosphate, 250 mM Mannitol, pH 7.5 | 7.0 | 2.1 | 34.4 | 92 |
| 30 mM Phosphate, 250 mM Mannitol, pH 7.5 | 7.3 | 3.1 | 50.2 | 64 |
| CHT, 42.5% B (diafiltrated) | 1.0 | 4.2 | 56.3 | 141 |
| CHT, 42.5% B (diafiltrated) | 5.0 | 10.7 | 48.5 | 149 |
| CHT, 42.5% B (diafiltrated) | 3.0 | 6.6 | 41.1 | 121 |
| CHT, 42.5% B (diafiltrated) + 100 Mannitol | 2.0 | 4.6 | 44.4 | 119 |
| CHT, 40% B (eluted) | 2.6 | 2.8 | 45 | 143 |
| CHT, 40% B (eluted) | 3.0 | 6.7 | 41.9 | 139 |
| CHT, 47.5% B (eluted) | 2.6 | 15.6 | 48.8 | 132 |
| CHT, 47.5% B (eluted) | 2.6 | 11.7 | 53.1 | 132 |
| CHT, 33% B (diafiltrated) | 5.0 | 23.1 | 43.9 | 140 |
| CHT, 47.5% B (diafiltrated) | 3.1 | 12.7 | 47 | 132 |
| 88 mM Phosphate, 20 mM Mannitol, pH 7 | 2.1 | 18.4 | 43.7 | 96 |
| | <5% | <10% | <40% | 100 ± 10 |

### EPT Powder Characterization

To measure residual moisture, a 5.9-mg portion of dried powder was incubated at 70° C. for 16 h, and the weight difference before and after heating was used to calculate moisture content. To determine solubility, dried powder was weighed and a calculated volume of milli Q water was added to reconstitute EPT powder to 1 mg/mL. The concentration of the reconstitute solution was measured by Nanodrop (Thermo Fisher Scientific; Waltham, MA, USA) using an extinction coefficient at A280 of 0.7857. The percent solubility was calculated based on the difference in 1 mg/mL versus the determined nanodrop concentration.

Percent monomer was determined by size-exclusion high performance liquid chromatography (SEC-HPLC). SEC-HPLC was run as previously described [17]. Briefly, reconstituted EPT at 1 mg/mL was applied to a Tosoh TSKgel SuperSW3000 column using 100 mM sodium phosphate, pH 7.2, 150 mM sodium chloride running buffer. EPT standard (a bulk solution prepared in PBS before spray drying) was used as a control.

### GM1-Capture KDEL-Detection (GM1/KDEL)-ELISA

The assay was done as described in Morris et al. [24]. Plates were coated with 100 μL per well of 2 μg/mL GM1 ganglioside (Sigma Aldrich; St. Louis, MO, USA) diluted in a coating solution consisting of 3 mM sodium azide, 15 mM sodium carbonate, 35 mM sodium bicarbonate, pH 9.6. After overnight incubation (16 to 18 h) at 4° C., plates were washed three times with PBST (0.05% Tween 20 in 1×PBS) and blocked with a blocking solution (5% non-fat dry milk, 0.05% Tween 20 in 1×PBS) for 1 h at room temperature, then washed with PBST thrice. Three-fold serially diluted, duplicate samples (100 μL/well) were added to plates in 1% PBSTM (1% dry milk, 0.05% Tween 20 in 1×PBS). Samples were incubated on plates for 1 h at 37° C. Plates were washed and mouse anti-KDEL monoclonal antibody (Enzo Life Sciences; Farmingdale, NY, USA) diluted 1:1,000 in 1% PBSTM (100 μL/well) was added; plates were then incubated at 37° C. for 1 h. Plates were washed and goat anti-mouse IgG-HRP (Southern Biotech; Birmingham, AL, USA) diluted 1:5,000 in 1% PBSTM was added, followed by incubation at 37° C. for 1 h. Plates were washed a final time and developed with 3,3′,5,5′-tetramethylbenzidine substrate (TMB). The reaction was stopped with 2 N sulfuric acid and the absorbance at 450 nm was immediately measured with a BioTek plate reader.

### EPT Capsule Package and Preparation

Torpac size M gelatin capsules were packed with 2.3-2.8 mg dried EPT product per capsule (corresponding to 5±1 μg of EPT) using the ProFunnel capsule filling system (Torpac; Fairfield, NJ, USA). Capsule cap and body joints were painted with 4% Eudragit L100 anionic polymer coating solution and allowed to dry for 20 min. Capsules were loaded into a size M capsule holder and dipped just past the cap and body joints into 4% Eudragit L100 coating solution (recipe recommended by Torpac) and allowed to dry for 25 min according to the capsule manufacturer instructions. Capsules were flipped and reloaded into the holder and dipped past the cap and body joints into 4% Eudragit L100 coating solution. Dipping of capsules was repeated with 20% Eudragit L100 coating solution. Eudragit L100 was the chosen polymer due to its degradation at pH 6.8, the approximate pH of the proximal colon.

### Capsule Disintegration Test

Individual EPT-containing enteric-coated capsules (n=5) were submerged in 1 N hydrochloric acid for 2 h at room temperature. Acid submerging each capsule was removed and stored separately. Capsules were washed briefly with sodium phosphate buffer (pH 6.0) followed by submersion in sodium phosphate buffer (pH 6.8). Aliquots were removed from each tube following light vortexing at 5, 10, 15, 30, 60, 90, 120, and 180 min and stored individually. EPT release from individual capsules was analyzed by CTB sandwich ELISA.

### CTB Sandwich ELISA

The concentration of EPT standard was measured by Nanodrop (Thermo Fisher Scientific; Waltham, MA, USA). Plates were coated with 100 μL per well of 2.5 μg/mL of the rat anti-CTB monoclonal antibody 7A12B3 diluted in PBS. After overnight incubation (16 to 24 h) at 4° C., plates were washed three times with PBST (0.05% Tween 20 in 1×PBS) and blocked with a blocking solution (3% bovine serum albumin, 0.05% Tween 20 in 1×PBS) for 2 h at room temperature, then washed with PBST in triplicate. Three-fold serially diluted, duplicate standard samples (100 μL/well) were added to plates in dilution buffer (1% bovine serum albumin, 0.05% Tween 20 in 1×PBS). Unknown samples were diluted 1:10 in dilution buffer and added to plates in duplicate (100 μL/well). Samples were incubated on plates for 1 h at room temperature. Plates were washed and rabbit anti-CTB polyclonal antibody (Abcam; Cambridge, UK) diluted 1:20,000 in dilution buffer (100 μL/well) was added; plates were then incubated at room temperature for 1 h. Plates were washed and goat anti-rabbit IgG-HRP (Southern Biotech; Birmingham, AL, USA) diluted 1:100,000 in dilution buffer was added, followed by incubation at room temperature for 1 h. Plates were washed a final time and developed with TMB. The reaction was stopped with 2 N sulfuric acid and the absorbance at 450 nm was immediately measured with a BioTek plate reader. Percent release was determined by extrapolation of calculated EPT concentrations using CTB sandwich ELISA compared to a known fixed mass of 5 μg EPT per capsule.

### Acute DSS Colitis Model and EPT Treatment

Groups of 10 female C57BL/6 mice, randomly assigned, were used. 3% (w/v) DSS (M.W. 36,000-50,000; M.P. Biomedicals; Santa Ana, CA, USA) was administered in drinking water ad libitum for 7 days. Body weights were monitored daily from the start of DSS exposure to sacrifice on day 14. On the last day of DSS exposure, animals were orally gavaged 100 μL PBS, 100 μL of EPT powder dissolved in PBS (0.03 mg/mL solution) after administration of sodium bicarbonate (200 μL of 30 mg/mL solution) as described previously, or enteric coated capsules filled with 5 μg EPT (described above) [15]. Animals recovered with normal drinking water for 7 days. Disease activity index (DAI) scores, consisting of body weight loss, fecal consistency and occult blood tests, were recorded following sacrifice and performed as previously described [25]. Distal colon tissues were fixed in neutral buffered formalin and stained with hematoxalin and eosin (H&E). Histopathological scores, a combination score comprised of crypt architecture, inflammatory infiltrate, muscle thickening and goblet cell presences scores, were determined as previously described [24,25]. Each category was ranked on a scale from 0 to 3 and summed to obtain a single histopathological damage score for each tissue.

### Statistics

For all data, outliers were determined by statistical analysis using the Grubb's test and excluded from further analysis if p<0.05. Graphs were prepared and analyzed using Graphpad Prism version 5.0 (Graphpad Software; La Jolla, CA, USA). To compare two data sets, an unpaired, two-tailed Student's t test was used. To compare three or more data sets, one-way ANOVA with Bonferroni's multiple-comparison post-test.

### Example 2. Pre-Formulation Analysis

Buffers and excipients were screened to determine a combination producing optimal EPT powder. This was determined by assessing pentamer disassembly into monomer, residual moisture, yield, and solubility of powder in water, with a target product profile (TPP) of <5% monomer, <10% moisture, and 100±10% solubility (Table 1). Of the buffers tested, PBS and PBS+100 mM mannitol were chosen from the screened buffers based on the TPP values set for the aforementioned parameters. To assess stability of the chosen buffers, pentamer degradation and water-solubility of EPT formulated in PBS or PBS+100 mM mannitol were analyzed over a period of three weeks (Table 2). The screened and finalized drying conditions are summarized in Table 3. Given the importance of pentamer stability to the epithelial repair activity of EPT [20], PBS+100 mM mannitol was used to optimize drying conditions and subsequent experiments. A variety of inlet and outlet temperature range combinations were assessed to determine which would result in optimal pentamer stability. A lower inlet temperature range (116-122° C.) and higher outlet temperature range (64-67° C.) were found to result in the lowest degree of pentamer degradation. Therefore, these conditions were utilized for subsequent batch productions.

TABLE 2
Stability of EPT powder in chosen buffer excipients

| | % | % | % | % |
|---|---|---|---|---|
| Buffer | Monomer | Moisture | Loss | Solubility |
| PBS | 4.9 | 2.9 | 29.3 | 106 |
| PBS, 100 mM Mannitol | 3.1 | 0 | 60.4 | 97 |
| CHT, 42.5% B (diafiltrated) | 3.0 | 6.6 | 41.1 | 121 |
| CHT, 42.5% B (diafiltrated) + 100 Mannitol | 2.0 | 4.6 | 44.4 | 119 |

1 Week

| | | |
|---|---|---|
| PBS | 5.9 | 110 |
| PBS, 100 mM Mannitol | 4.0 | 97 |
| CHT, 42.5% B (diafiltrated) | 3.6 | 132 |
| CHT, 42.5% B (diafiltrated) + 100 Mannitol | 2.3 | 114 |

2 Weeks

| | | |
|---|---|---|
| PBS | 4.8 | 110 |
| PBS, 100 mM Mannitol | 3.3 | 99 |
| CHT, 42.5% B (diafiltrated) | 2.8 | 137 |
| CHT, 42.5% B (diafiltrated) + 100 Mannitol | 3.0 | 110 |

3 Weeks

| | | |
|---|---|---|
| PBS | 5.9 | 110 |
| PBS, 100 mM Mannitol | 3.4 | 99 |
| CHT, 42.5% B (diafiltrated) | 3.5 | 137 |
| CHT, 42.5% B (diafiltrated) + 100 Mannitol | 3.0 | 110 |

TABLE 3
Optimization of spray-dry parameters

| | % | % | | % | | | | | |
|---|---|---|---|---|---|---|---|---|---|
| Buffer | Monomer | Moisture | % Loss | Solubility | Q-Flow | Inlet | Outlet | Aspirator | Pump |
| PBS | 12.3 | 9.7 | 36.2 | 97 | — | 121 | 66-67 | 90 | 20 |
| PBS | 5.9 | 4.5 | 34.5 | 101 | — | 137-140 | 63-65 | 80 | 30 |
| PBS | 3.2 | 32.3 | 29.3 | 101 | — | 119-128 | 61-67 | 90 | 24 |
| PBS | 8.0 | 3.7 | 36.2 | 99 | — | 120 | 61-64 | 90 | 20 |
| PBS | 4.9 | 2.9 | 29.3 | 106 | 35 | 118-125 | 61-65 | 90 | 20 |
| PBS | 11 | — | 51.7 | 99 | 34 | 121-123 | 61-63 | 90 | 20 |
| PBS, 100 mM Mannitol | 4.4 | 3.9 | 60.4 | 101 | | 137-140 | 63-66 | 80 | 30 |
| PBS, 100 mM Mannitol | 3.1 | 0 | 60.4 | 97 | 35 | 120-123 | 62-64 | 90 | 20 |
| PBS, 100 mM Mannitol | 1.0 | 9.5 | 54.2 | 97 | 35 | 116-122 | 64-67 | 95 | 20 |
| PBS, 100 mM Mannitol @ 5 mg/ml | 1.1 | 4.9 | 69.8 | 96 | 35 | 118-122 | 61-63 | 90 | 20 |
| PBS, 100 mM Mannitol | 9 | — | 55 | 119 | 34 | 121-124 | 63-66 | 90 | 20 |

*Finalized drying conditions

### Example 3. Stability and Disintegration Testing of EPT Capsules

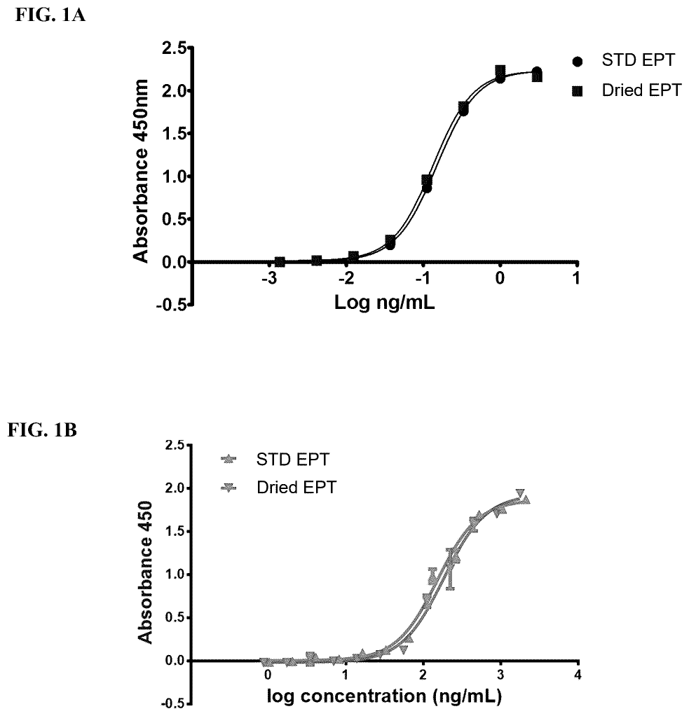

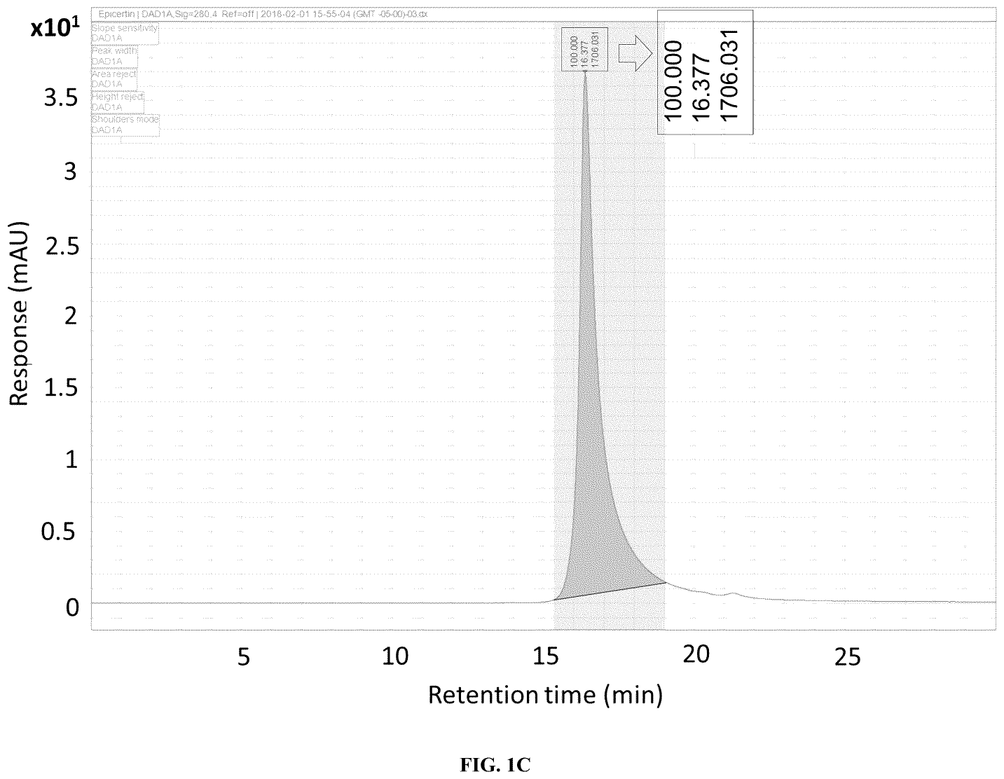

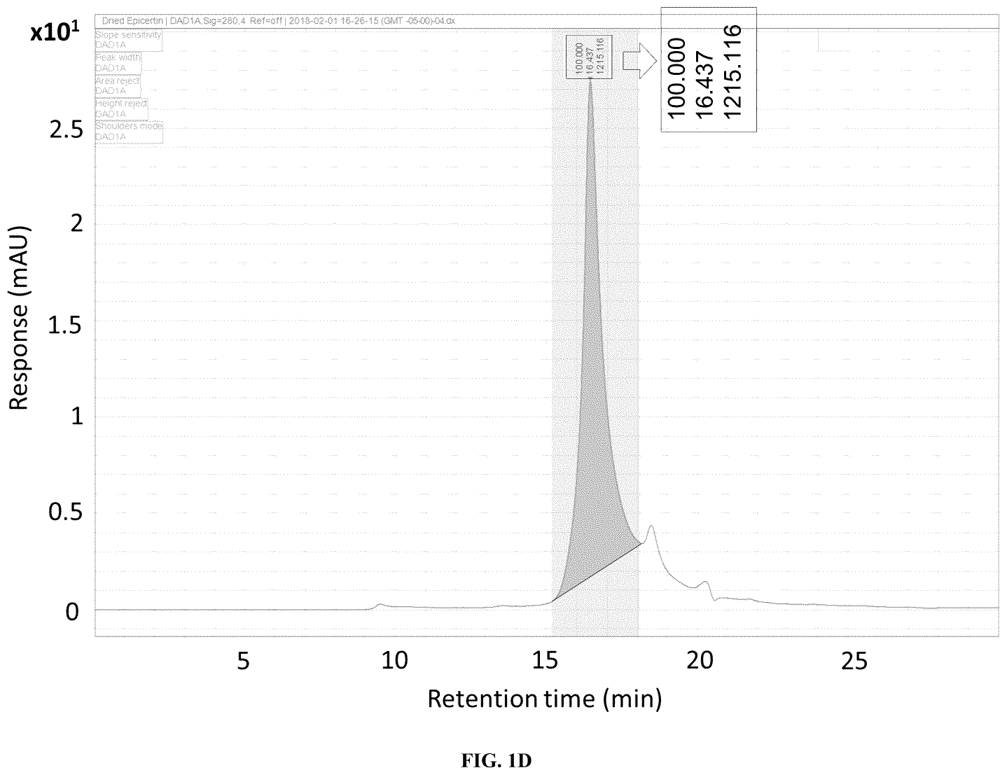

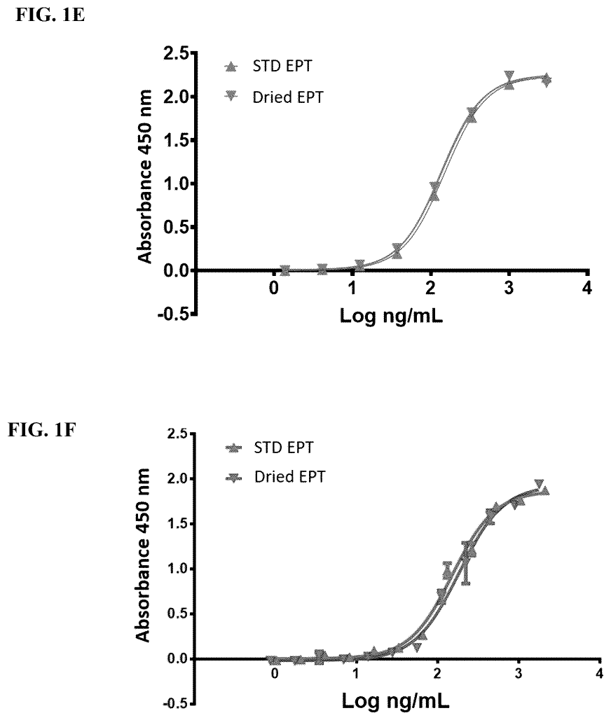

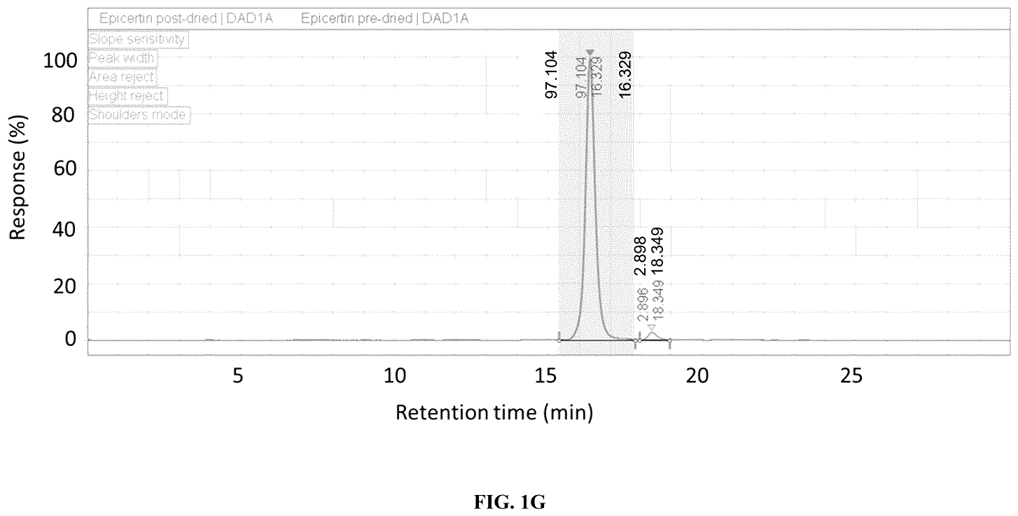

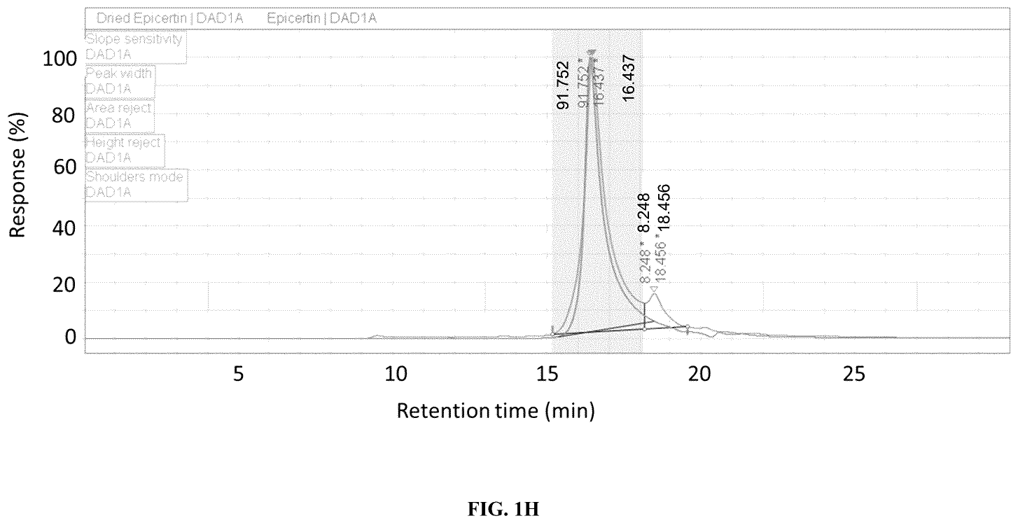

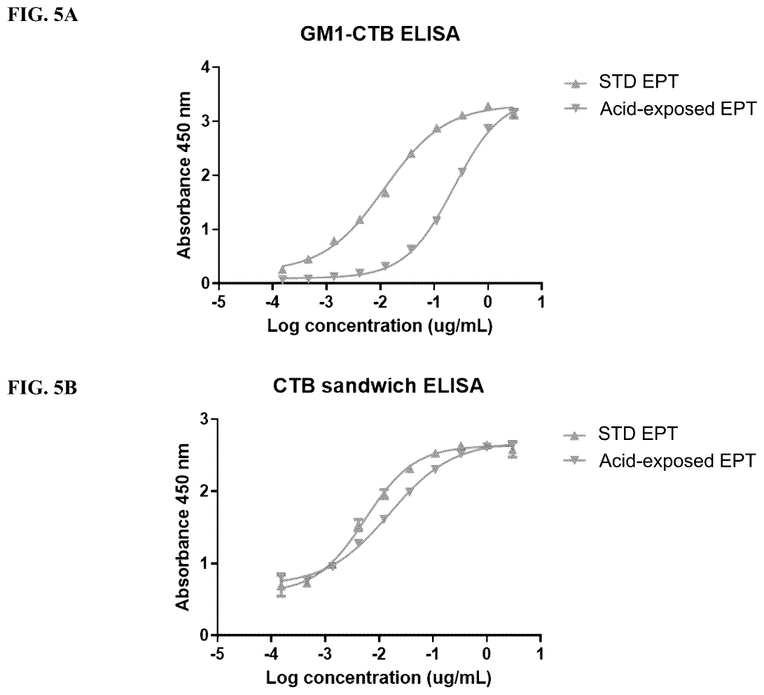

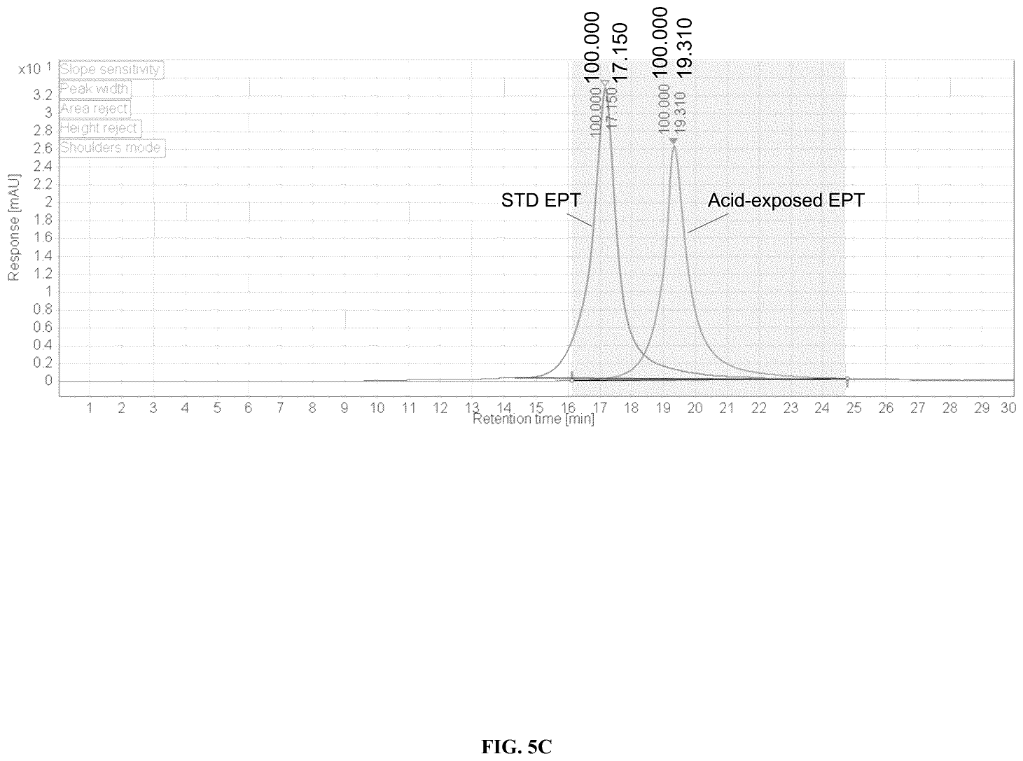

To demonstrate the stability of the chosen prototype EPT powder immediately after spray-drying, a GM1/KDEL-ELISA and SEC-HPLC were performed to detect the presence of intact KDEL sequence and the conformational state of CTB pentamer, which are crucial for the mucosal healing activity of EPT. This immunoassay and analytical analysis were repeated at 9 months post-spray dry with EPT powder stored under desiccation at 23° C. in parafilm-wrapped tubes. GM1/KDEL-ELISA results demonstrate that GM1 binding affinity of spray dried EPT was unchanged after drying (FIGS. 1A and 1E) and 9 months post-drying when stored under desiccation at 23° C. (FIGS. 1B and 1F). SEC-HPLC chromatograms illustrate the stability of EPT pentamers in 9 months post-drying (FIGS. 1C, 1D, 1G and 1H). The small peak of 8.2% at a retention time of ˜18.5 min correlates to EPT monomers (see FIG. 5C), indicating only slight pentamer degradation after 9 months post-drying stored under desiccation at 23° C. (FIGS. 8D and 8I).

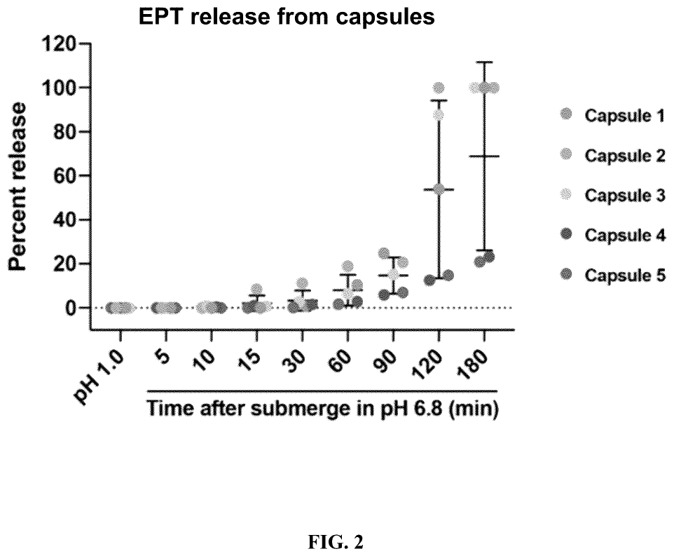

Following packing of EPT powder into gelatin capsules and coating with an enteric coating solution, pH-dependent release of EPT was assessed by a capsule disintegration assay. Capsules were coated with Eudragit S100 coating solution to prevent the release of EPT prior to arrival at the proximal colon where the intraluminal pH has increased to ˜ pH 6.8 after transiting through the stomach and small intestine [26,27]. The capsule disintegration assay simulated passage of the capsule through gastric acid and allowed analysis of the release profile of EPT following exposure to pH 6.8. Recovery of EPT from capsules was determined by a CTB detection sandwich ELISA, which has the capacity to detect both GM1-binding pentamer and disassembled CTB molecular species unlike GM1-capture ELISA (FIGS. 5A-5C), as EPT dissociation could occur if the capsule content was prematurely discharged and exposed to low pH conditions (FIG. 5C). The data revealed that EPT was released from the polymer-coated capsules in a pH-dependent manner (FIG. 2). No EPT was detectable at pH 1.0, suggesting that the enteric coating prevented the release of EPT from capsules prior to expected release at pH 6.8. The same coating solution and method was used to prepare capsules used in the following acute colitis model.

### Example 4. Efficacy of EPT Enteric-Coated Capsules in an Acute Colitis Model

It has been demonstrated that oral administration of EPT mitigates mucosal damage and inflammation in preclinical colitis and oral mucositis models. For the preclinical colitis study, a liquid EPT formulation was administered orally after neutralization of gastric acid by pre-administration of a large volume of bicarbonate containing solution because the protein is acid labile.

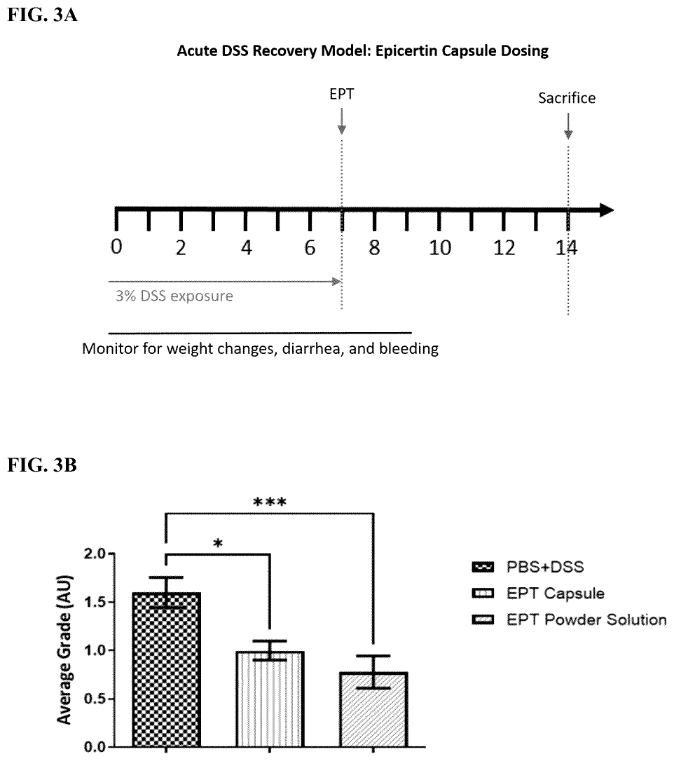

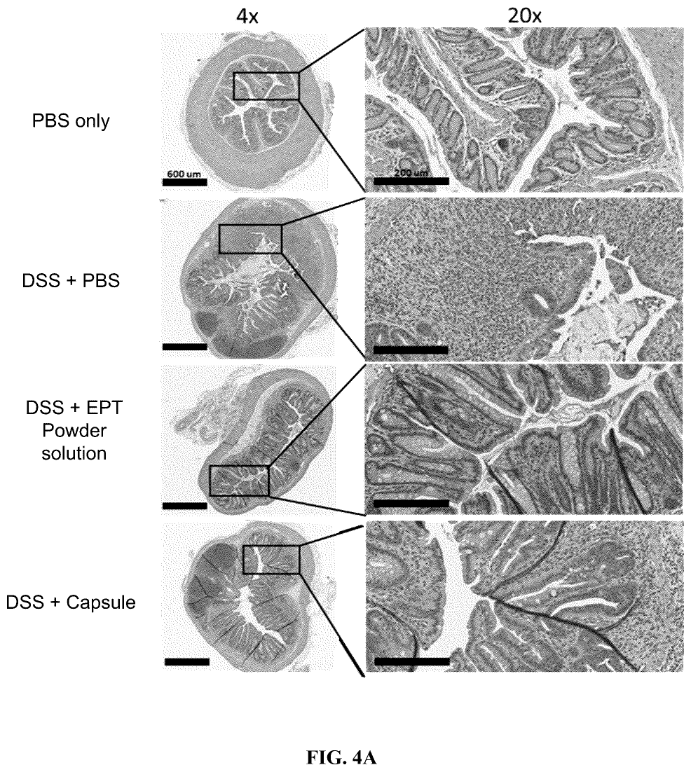

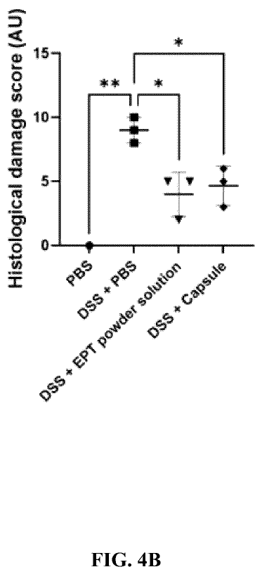

The efficacy of EPT delivered by this oral capsule formulation was assessed compared to standard EPT in an acute DSS colitis mouse model [15,20]. In this model, animals were exposed to 3% DSS ad libitum in drinking water for 7 days at which time mice were dosed 5 μg resuspended dried EPT (EPT powder solution) or PBS via oral gavage after administration of sodium bicarbonate. A third group of mice were dosed with an enteric-coated capsule containing 5 μg EPT with no administration of sodium bicarbonate. All animals were monitored an additional 7 days following DSS cessation. Compared to PBS, DAI scores were decreased in mice administered a EPT capsule directly as well as sodium bicarbonate followed by reconstituted EPT powder (p<0.05 and p<0.001, respectively) (FIGS. 3A-3B). To corroborate the aforementioned results, we performed a histopathological evaluation to assess the presence of hallmark colitis markers, such as alterations in crypt height and loss, epithelial barrier disruption, and immune cell infiltration, in hematoxalin and eosin (H&E) stained tissues. EPT powder solution administration following gastric acid neutralization and encapsulated EPT similarly protected mice from DSS-induced acute colitis. Treatment with EPT by capsule administration or oral gavage following gastric acid neutralization protected mice from crypt loss and distortion, inflammatory cell infiltrates, muscle thickening, and goblet cell loss (FIGS. 4A-4B). However, few crypt structural alterations, primarily crypt branching, were noted in tissues from both EPT treatment groups. Taken together, these results support equivalence between EPT treatment in solution following acid neutralization and encapsulated EPT treatment.

EPT is a variant of the nontoxic component of the cholera toxin that exhibits unique mucosal healing activity in the colon [16,18, 21]. Previous studies examining the therapeutic potential of EPT in mouse colitis models have primarily focused on one route of administration: oral gavage. An issue with this route of administration, however, is the need to neutralize gastric acid with sodium bicarbonate prior to gavage as CTB is acid labile. This is a drawback when moving forward with development of EPT as a therapeutic for UC and determining a final drug product formulation. Although oral agents are typically preferred by patients undergoing treatment for chronic diseases such as UC, gastric acid neutralization requirements could potentially affect patient outcomes by lowering adherence and ease of administration. Therefore, a prototype solid oral formulation that would allow EPT to circumvent gastric acid degradation and allow for topical administration to affected areas was developed. Described herein is an encapsulated spray-dried drug substance coated with an enteric coating to allow for pH-dependent release of EPT at the colon.

A major technical advance made in the present study towards a solid oral formulation is the establishment of the method of drying the drug substance. Drying of pharmaceuticals is a long-implemented practice commonly used to enhance final drug product for a variety of purposes; examples of the benefits of biopharmaceutical dehydration include: handling and storage improvement, decrease in transportation cost, improved stability and aid in development of modified or delayed release particles [28,29]. It is known that proteins are more stable in solid rather than liquid form [30-33]. Use of solid formulations can greatly increase shelf-life and reduce storage regulations, saving patients and manufacturers money in lost production costs due to expired product. Further, oral capsules filled with dried protein may be coated with a time- or pH-dependent coating to allow for targeted release in the GI tract [29]. This is especially useful when administering CTB orally as it allows for release at the affected site. Without this coating, orally administered pentameric CTB would degrade into nonfunctional monomers upon exposure to the stomach acid. Dehydrating CTB is one solution to this issue.

Previously, CTB has been dehydrated by a variety of methods. A freeze-dried inactivated whole-cell oral cholera vaccine was formulated in attempts to optimize delivery of mass quantities of vaccine to low-income countries [34]. This formulation elicited strong serum and gut mucosal anti-LPS antibody responses in immunized mice; these responses were comparable to those achieved with equivalent liquid formulation [34]. The dry formulation is beneficial in substantially reducing package volumes and weights when delivering product to areas in need of mass vaccination. Further, CTB has been successfully spray-dried in the form of heat-killed Vibrio cholerae-containing microparticles [35]. The benefits of the spray-drying process with EPT was utilized to develop a more optimal oral formulation of the protein. When a protein is spray-dried, conditions need to be tailored to the protein being dried since materials undergo some thermal stress which can result in protein degradation; hence, identification of ideal heating conditions is critical. CTB pentamer degradation occurs approximately between 66° C. and 78° C. [36-38]. Here, outlet temperatures range between 61° C. and 67° C. to maintain stability of functional EPT pentamer were screened. Since optimal TPP parameters were achieved with outlet temperatures closer to 67° C., testing outlet temperatures closer to 78° C. could possibly result in a further improved powder by solubility or moisture content. The most relevant source of stress during spray-drying results from the dehydration process, therefore the addition of excipients to the liquid solution prior to spray drying is crucial to replace the hydrogen bonding that exists in an aqueous environment [29]. In this study, a screen was developed to identify optimal excipient conditions to improve target profile parameters.

A buffer and excipient screen to produce an ideal dried EPT powder based on a set TPP (<5% monomer, <10% moisture, and 100±10% solubility). Addition of mannitol decreased the presence of EPT monomer from approximately 5% to 3% (Tables 2 and 3). As such, addition of mannitol, which is thermostable [39-41], improved stability of EPT pentamer. Because PBS+100 mM mannitol outperformed all other screened buffers in the aforementioned TPP categories, it was therefore chosen as the formulation buffer. All TPP categories were met, however, loss of drug product was a consistent problem as 70% was the maximum recovery among all buffers tested (data not shown). Other studies using this particular spray drier consistently report yields below 50% [42]. Although one issue with this instrument is in aspects of the design, another manageable issue is identifying an ideal inlet to outlet temperature ratio for the protein of interest [42].

Stability of EPT powder produced in the chosen buffer was confirmed by measuring monomer content and percent solubility each week for a total of three weeks (Tables 2 and 3). Low hygroscopicity of mannitol likely had a positive impact on moisture content; it is known that mannitol is an ideal excipient to minimize moisture in a dried formulation [40,41]. Monomer content and solubility remained stable around 3% and 98%, respectively, over the course of three weeks. Mannitol seems to have a protective effect on EPT pentamers, possibly due its thermostability. Other thermoprotective agents, such as dextrose, trehalose and lactose, may also serve as an excipient. Lactose is the most commonly utilized excipient in spray drying [39,43]. mannitol and lactose are both attractive excipients as they are soluble in water and are non-toxic. Lactose has an advantage to mannitol as it is more economical, although it should be noted that lactose has a higher hygroscopicity which can hinder stability of the dried product [39,41]. Further, lactose is more likely to react with active pharmaceutical ingredients as it is a reducing sugar, whereas mannitol exhibits a strong inertness [39].

Upon selection of buffer composition, spray dry parameters were further investigated. Spray dry parameters were optimized by screening a combination of inlet and outlet temperatures to determine a combination producing EPT powder with the best possible TPP values (Table 3). It has been suggested that a high inlet temperature to outlet temperature ratio might be the key to maximizing yield. Our results are consistent with this claim as the highest inlet:outlet temperature condition tested achieved optimal TPP values (Table 3). We also demonstrated via SEC-HPLC and GM1-KDEL detection ELISA that spray dried EPT maintains GM1 binding affinity and remains stable under dry conditions for up to 9 months (FIGS. 1A-1H). This combination of factors indicates that EPT powder encapsulated immediately post-dry and after 9 months should exhibit similar effects upon administration.

The oral formulation presented here was designed for direct delivery of EPT to the target site by utilizing an anionic polymer coating that makes pH-dependent release at the colon possible. The in vitro disintegration test suggested that not all capsules released 100% of encased EPT (FIG. 2). It is possible that there are inconsistencies in the thickness of the enteric coating around the capsules, leading to variations in release profiles. The combination of dipping capsules by and viscosity of the solution could lead to disparities between capsule coatings. This could be ameliorated by coating the capsules using an automated system that would likely be utilized when coating capsules at an industrial scale. Further, it was observed during disintegration testing that capsules tend to fold in on themselves when free-floating in solution. This is an unlikely issue in vivo as movement through the GI tract would prevent the folding over of capsules. In support of this hypothesis, the acute DSS study showed that the enteric-coated EPT capsule ameliorated acute DSS-induced colitis, indicating that the protein was successfully released from the capsules at the site of mucosal damage (FIGS. 3A-4B). The histopathological results described here are also consistent with previous findings in acute and chronic DSS colitis studies evaluating EPT treatment [16,17,21]. Taken together, these results show that treatment using the capsule formulation described herein does not require gastric acid neutralization, and would theoretically ease difficulty of administration and boost patient adherence in UC patients.

EPT is a recombinant variant of the cholera toxin B subunit, modified with a C-terminal KDEL endoplasmic reticulum retention motif. EPT has therapeutic potential for ulcerative colitis treatment. Previously, orally administered EPT demonstrated colon epithelial repair activity in dextran sodium sulfate (DSS)-induced acute and chronic colitis in mice. However, the oral dosing requires cumbersome pretreatment with sodium bicarbonate to conserve the acid-labile drug substance while transit through the stomach, hampering its facile application in chronic disease treatment. Here, we developed a solid oral formulation of EPT that circumvents degradation in gastric acid. EPT was spray-dried and packed into enteric-coated capsules to allow for pH-dependent release in the colon. A GM1-capture KDEL-detection ELISA and size-exclusion HPLC indicated that EPT powder maintains activity and structural stability for up to 9 months. Capsule disintegration tests showed that EPT remained encapsulated at pH 1 but was released over 180 min at pH 6.8, the approximate pH of the proximal colon. An acute DSS colitis study confirmed the therapeutic efficacy of encapsulated EPT in C57BL/6 mice upon oral administration without gastric acid neutralization pretreatment compared to vehicle-treated mice (p<0.05). These results provide a foundation for an enteric-coated oral formulation of spray-dried EPT.

Dry formulation has several advantages for storage, drug compounding, drug delivery system development. The development and demonstration of this technology is another critical step towards commercialization of EPT for human therapeutic use. No products currently available for UC or in development possess the combination of attributes that this product would offer. It is anticipated that the combination of EPT's unique wound healing effect and the convenience of a solid dosage form will represent a highly desired “first in class” treatment with the potential to transform UC standard of care.

The developed dry formulated EPT allows for effortless formulation for targeted delivery, ease of administration and higher patient acceptance, which will in turn help assure higher treatment compliance in chronic therapy scenarios.

While example embodiments have been particularly shown and described, it will be understood by those skilled in the art that various changes in form and details may be made therein without departing from the scope of the embodiments encompassed by the appended claims.

### Sequences

A nucleic acid sequence of a wild-type cholera toxin B subunit from Vibrio cholerae:

(SEQ ID NO: 1)
accccacaaaacatcactgacttgtgtgctgagtaccacaacacccaaa
tccacaccctcaatgacaagatctttagctacaccgagagccttgctgg
caagagggagatggctatcatcaccttcaagaatggtgctaccttccaa
gtggaggtgcctggaagccaacacattgatagccaaaagaaggccattg
agaggatgaaggacacacttaggatagcttacctcactgaggctaaggt
ggagaagctttgtgtgtggaacaacaagaccccccatgctattgctgcc
atcagcatggccaac.

An amino acid sequence of a wild-type cholera toxin B subunit from Vibrio cholerae.

(SEQ ID NO: 2)
TPQNITDLCAEYHNTQIHTLNDKIFSYTESLAGKREMAIITFKNGATFQ
VEVPGSQHIDSQKKAIERMKDTLRIAYLTEAKVEKLCVWNNKTPHAIAA
ISMAN.

A nucleic acid sequence encoding a cholera toxin B subunit variant polypeptide modified to include a C-terminal endoplasmic reticulum signal and to include no N-linked glycosylation sequons at Asn4:

(SEQ ID NO: 3)
accccacaaagcatcactgacttgtgtgctgagtaccacaacacccaaa
tccacaccctcaatgacaagatctttagctacaccgagagccttgctgg
caagagggagatggctatcatcaccttcaagaatggtgctaccttccaa
gtggaggtgcctggaagccaacacattgatagccaaaagaaggccattg
agaggatgaaggacacacttaggatagcttacctcactgaggctaaggt
ggagaagctttgtgtgtggaacaacaagaccccccatgctattgctgcc
atcagcatggccaactccgagaaggatgaactc.

An amino acid sequence of a cholera toxin B subunit variant polypeptide modified to include a C-terminal endoplasmic reticulum signal and to include no N-linked glycosylation sequons at Asn4:

(SEQ ID NO: 4)
TPQSITDLCAEYHNTQIHTLNDKIFSYTESLAGKREMAIITFKNGATFQ
VEVPGSQHIDSQKKAIERIVIKDTLRIAYLTEAKVEKLCVWNNKTPHAI
AAISMANSEKDEL.

A nucleic acid sequence encoding a cholera toxin B subunit variant polypeptide modified to include a C-terminal endoplasmic reticulum retention signal and having one N-linked glycosylation sequon at Asn4:

(SEQ ID NO: 5)
accccacaaaacatcactgacttgtgtgctgagtaccacaacacccaaa
tccacaccctcaatgacaagatctttagctacaccgagagccttgctgg
caagagggagatggctatcatcaccttcaagaatggtgctaccttccaa
gtggaggtgcctggaagccaacacattgatagccaaaagaaggccattg
agaggatgaaggacacacttaggatagcttacctcactgaggctaaggt
ggagaagctttgtgtgtggaacaacaagaccccccatgctattgctgcc
atcagcatggccaactccgagaaggatgaactc.

An amino acid sequence of a cholera toxin B subunit variant polypeptide modified to include a C-terminal endoplasmic reticulum retention signal and having one N-linked glycosylation sequon at Asn4:

(SEQ ID NO: 6)
TPQNITDLCAEYHNTQIHTLNDKIFSYTESLAGKREMAIITFKNGATFQ
VEVPGSQHIDSQKKAIERMKDTLRIAYLTEAKVEKLCVWNNKTPHAIAA
ISMANSEKDEL.

A nucleic acid sequence encoding a cholera toxin B subunit variant polypeptide modified to include a C-terminal endoplasmic reticulum retention signal and to include two N-linked glycosylation sequons at Asn4 and Asn103:

(SEQ ID NO: 7)
accccacaaaacatcactgacttgtgtgctgagtaccacaacacccaaa
tccacaccctcaatgacaagatctttagctacaccgagagccttgctgg
caagagggagatggctatcatcaccttcaagaatggtgctaccttccaa
gtggaggtgcctggaagccaacacattgatagccaaaagaaggccattg
agaggatgaaggacacacttaggatagcttacctcactgaggctaaggt
ggagaagctttgtgtgtggaacaacaagaccccccatgctattgctgcc
atcagcatggccaacgttactaaggatgaactc.

An amino acid sequence of a cholera toxin B subunit variant polypeptide modified to include a C-terminal endoplasmic reticulum retention signal and to include two N-linked glycosylation sequons at Asn4 and Asn103:

(SEQ ID NO: 8)
TPQNITDLCAEYHNTQIHTLNDKIFSYTESLAGKREMAIITFKNGATFQ
VEVPGSQHIDSQKKAIERMKDTLRIAYLTEAKVEKLCVWNNKTPHAIAA
ISMANVTKDEL.

A nucleic acid sequence encoding another cholera toxin B subunit variant polypeptide modified to include a C-terminal endoplasmic reticulum retention signal and to include two N-linked glycosylation sequons at Asn4 and Asn21:

(SEQ ID NO: 9)
accccacaaaacatcactgacttgtgtgctgagtaccacaacacccaaa
tccacaccctcaatgacactatctttagctacaccgagagccttgctgg
caagagggagatggctatcatcaccttcaagaatggtgctaccttccaa
gtggaggtgcctggaagccaacacattgatagccaaaagaaggccattg
agaggatgaaggacacacttaggatagcttacctcactgaggctaaggt
ggagaagctttgtgtgtggaacaacaagaccccccatgctattgctgcc
atcagcatggccaactccgagaaggatgaactc.

An amino acid sequence of another cholera toxin B subunit variant polypeptide modified to include a C-terminal endoplasmic reticulum retention signal and to include two N-linked glycosylation sequons at Asn4 and Asn21:

(SEQ ID NO: 10)
TPQNITDLCAEYHNTQIHTLNDTIFSYTESLAGKREMAIITFKNGATFQ
VEVPGSQHIDSQKKAIERMKDTLRIAYLTEAKVEKLCVWNNKTPHAIAA
ISMANSEKDEL.

A nucleic acid sequence encoding a cholera toxin B subunit variant polypeptide modified to include a C-terminal endoplasmic reticulum retention signal and to include three N-linked glycosylation sequons at Asn4, Asn21, and Asn103:

(SEQ ID NO: 11)
accccacaaaacatcactgacttgtgtgctgagtaccacaacacccaaa
tccacaccctcaatgacactatctttagctacaccgagagccttgctgg
caagagggagatggctatcatcaccttcaagaatggtgctaccttccaa
gtggaggtgcctggaagccaacacattgatagccaaaagaaggccattg
agaggatgaaggacacacttaggatagcttacctcactgaggctaaggt
ggagaagctttgtgtgtggaacaacaagaccccccatgctattgctgcc
atcagcatggccaacgttactaaggatgaactc.

An amino acid sequence of a cholera toxin B subunit variant polypeptide modified to include a C-terminal endoplasmic reticulum retention signal and to include three N-linked glycosylation sequons at Asn4, Asn21, and Asn103:

(SEQ ID NO: 12)
TPQNITDLCAEYHNTQIHTLNDTIFSYTESLAGKREMAIITFKNGATFQ
VEVPGSQHIDSQKKAIERIVIKDTLRIAYLTEAKVEKLCVWNNKTPHAI
AAISMANVTKDEL.

A nucleic acid sequence encoding another cholera toxin B subunit variant polypeptide modified to include a C-terminal endoplasmic reticulum retention signal and to include three N-linked glycosylation sequons at Asn4, Asn21, and Asn103:

(SEQ ID NO: 13)
accccacaaaacatcactgacttgtgtgctgagtaccacaacacccaaa
tccacaccctcaatgacactatctttagctacaccgagagccttgctgg
caagagggagatggctatcatcaccttcaagaatggtgctaccttccaa
gtggaggtgcctggaagccaacacattgatagccaaaagaaggccattg
agaggatgaaggacacacttaggatagcttacctcactgaggctaaggt
ggagaagctttgtgtgtggaacaacaagaccccccatgctattgctgcc
atcagcatggccaacgttactggtggtggaggatccgagaaggatgaac
tc.

An amino acid sequence of another cholera toxin B subunit variant polypeptide modified to include a C-terminal endoplasmic reticulum retention signal and to include three N-linked glycosylation sequons at Asn4, Asn21, and Asn103:

(SEQ ID NO: 14)
TPQNITDLCAEYHNTQIHTLNDTIFSYTESLAGKREMAIITFKNGATFQ
VEVPGSQHIDSQKKAIERMKDTLRIAYLTEAKVEKLCTPHAIAAISMAN
VTGGGGSEKDEL.

A nucleic acid sequence encoding a cholera toxin B subunit variant polypeptide with an N-terminal secretory signal from Vibrio cholerae and a C-terminal endoplasmic reticulum retention signal:

(SEQ ID NO: 15)
atggctatcaagctcaagtttggagtgttcttcactgtgctecttagct
ctgcctatgcacatggcaccccacaaaacatcactgacttgtgtgctga
gtaccacaacacccaaatccacaccctcaatgacaagatctttagctac
accgagagccttgctggcaagagggagatggctatcatcaccttcaaga
atggtgctaccttccaagtggaggtgcctggaagccaacacattgatag
ccaaaagaaggccattgagaggatgaaggacacacttaggatagcttac
ctcactgaggctaaggtggagaagctttgtgtgtggaacaacaagaccc
cccatgctattgctgccatcagcatggccaactccgagaaggatgaact
c.

An amino acid sequence of a cholera toxin B subunit variant polypeptide including an N-terminal secretory signal from Vibrio cholerae and a C-terminal endoplasmic reticulum retention signal:

(SEQ ID NO: 16)
MAIKLKFGVFFTVLLSSAYAHGTPQNITDLCAEYHNTQIHTLNDKIFSYT
ESLAGKREMAIITFKNGATFQVEVPGSQHIDSQKKAIERMKDTLRIAYLT
EAKVEKLCVWNNKTPHAIAAISMANSEKDEL.

A nucleic acid sequence encoding a rice alpha-amylase secretory signal peptide:

(SEQ ID NO: 17)
atggggaagcaaatggccgccctgtgtggctttctcctcgtggcgttgct
ctggctcacgcccgacgtcgcgcatggt.

An amino acid sequence of a rice alpha-amylase secretory signal peptide:

(SEQ ID NO: 18)
MGKQMAALCGFLLVALLWLTPDVAHG.

A nucleic acid sequence encoding a Nicotiana plumbagenifolia calreticulin secretory signal peptide:

(SEQ ID NO: 19)
atggctactcaacgaagggcaaaccctagctctctccatctaattactgt
attctctctgctcgtcgctgtcgtctcaggt

An amino acid sequence of a Nicotiana plumbagenifolia calreticulin secretory signal peptide:

(SEQ ID NO: 20)
MATQRRANPSSLHLITVFSLLVAVVSG.

A nucleic acid sequence encoding an apple pectinase secretory signal peptide: atggcattgaagacacagttgttgtggtcattcgtggttgtgttcgttgtgtccttcagtacaacttcatgctcaggt (SEQ ID NO:21).

An amino acid sequence of an apple pectinase secretory signal peptide:

(SEQ ID NO: 22)
MALKTQLLWSFVVVFVVSFSTTSCSG.

A nucleic acid sequence encoding a barley alpha-amylase secretory signal peptide: atggcgaacaaacacttgtccctctccctcttcctcgtcctccttggcctgtcggccagcttggcctcaggt (SEQ ID NO:23).

An amino acid sequence encoding a barley alpha-amylase secretory signal peptide:

(SEQ ID NO: 24)
MANKHLSLSLFLVLLGLSASLASG.

An amino acid sequence of a cholera toxin B subunit variant polypeptide including a Ser26-to-Cys and an Ala102-to-Cys mutation:

(SEQ ID NO: 25)
TPQNITDLCAEYHNTQIHTLNDKIFCYTESLAGKREMAIITFKNGATFQV
EVPGSQHIDSQKKAIERMKDTLRIAYLTEAKVEKLCVWNNKTPHAIAAIS
MCNSEKDEL.

An amino acid sequence of a cholera toxin B subunit variant polypeptide including a rice alpha-amylase N-terminal secretory signal peptide and a C-terminal endoplasmic reticulum retention signal peptide:

(SEQ ID NO: 26)
MGKQMAALCGFLLVALLWLTPDVAHGTPQNITDLCAEYHNTQIHTLNDKI
FSYTESLAGKREMAIITFKNGATFQVEVPGSQHIDSQKKAIERMKDTLRI
AYLTEAKVEKLCVWNNKTPHAIAAISMANSEKDEL.

An amino acid sequence of a cholera toxin B subunit variant polypeptide including a Nicotiana plumbagenifolia calreticulin N-terminal secretory signal peptide and a C-terminal endoplasmic reticulum retention signal peptide:

(SEQ ID NO: 27)
MATQRRANPSSLHLITVFSLLVAVVSGTPQNITDLCAEYHNTQIHTLNDK
IFSYTESLAGKREMAIITFKNGATFQVEVPGSQHIDSQKKAIERMKDTLR
IAYLTEAKVEKLCVWNNKTPHAIAAISMANSEKDEL.

An amino acid sequence of a cholera toxin B subunit variant polypeptide including an apple pectinase N-terminal secretory signal peptide and a C-terminal endoplasmic reticulum retention signal peptide:

(SEQ ID NO: 28)
MALKTQLLWSFVVVFVVSFSTTSCSGTPQNITDLCAEYHNTQIHTLNDKI
FSYTESLAGKREMAIITFKNGATFQVEVPGSQHIDSQKKAIERMKDTLRI
AYLTEAKVEKLCVWNNKTPHAIAAISMANSEKDEL.

An amino acid sequence of a cholera toxin B subunit variant polypeptide including a barley alpha-amylase N-terminal secretory signal peptide and a C-terminal endoplasmic reticulum retention signal peptide:

(SEQ ID NO: 29)
MANKHLSLSLFLVLLGLSASLASGTPQNITDLCAEYHNTQIHTLNDKIFS
YTESLAGKREMAIITFKNGATFQVEVPGSQHIDSQKKAIERMKDTLRIAY
LTEAKVEKLCVWNNKTPHAIAAISMANSEKDEL.

Amino acids of non-limiting examples of endoplasmic reticulum retention signal peptides, with or without a two-amino acid linker, SE: SEKDEL (SEQ ID NO: 30), KDEL (SEQ ID NO: 31), SEHDEL (SEQ ID NO: 32), and HDEL (SEQ ID NO: 33).

An amino acid sequence of a cholera toxin B subunit variant polypeptide including a rice alpha-amylase N-terminal secretory signal peptide and a C-terminal endoplasmic reticulum retention signal peptide:

(SEQ ID NO: 34)
MGKQMAALCGFLLVALLWLTPDVAHGTPQSITDLCAEYHNTQIHTLNDKI
FSYTESLAGKREMAIITFKNGATFQVEVPGSQHIDSQKKAIERMKDTLRI
AYLTEAKVEKLCVWNNKTPHAIAAISMANSEKDEL.

### REFERENCES

1. Zhang, Y. Z.; Li, Y. Y. Inflammatory bowel disease: Pathogenesis. World J. Gastroenterol. 2014, 20, 91-99.
2. The Facts about Inflammatory Bowel Diseases; Crohn's & Colitis Foundation: New York, NY, USA, 2014.
3. Lautenschlager, C.; Schmidt, C.; Fischer, D.; Stallmach, A. Drug delivery strategies in the therapy of inflammatory bowel disease. Adv. Drug Deliv. Rev. 2014, 71, 58-76.
4. Anwer, M. K.; Ahmed, M. M.; Aldawsari, M. F.; Alshahrani, S.; Fatima, F.; Ansari, M. N.; Rehman, N. U.; Al-Shdefat, R. I. Eluxadoline Loaded Solid Lipid Nanoparticles for Improved Colon Targeting in Rat Model of Ulcerative Colitis. Pharmaceuticals 2020, 13, 255.
5. Yadav, V.; Varum, F.; Bravo, R.; Furrer, E.; Basit, A. W. Gastrointestinal stability of therapeutic anti-TNFα IgG1 monoclonal antibodies. Int. J. Pharm. 2016, 502, 181-187.
6. Gallo, G.; Kotze, P. G.; Spinelli, A. Surgery in ulcerative colitis: When? How? Best Pract. Res. Clin. Gastroenterol. 2018, 32-33, 71-78.
7. Fell, J. M.; Muhammed, R.; Spray, C.; Crook, K.; Russell, R. K. Management of ulcerative colitis. Arch. Dis. Child. 2016, 101, 469-474.
8. Danese, S.; Fiorino, G.; Peyrin-Biroulet, L. Positioning Therapies in Ulcerative Colitis. Clin. Gastroenterol. Hepatol. 2020, 18, 1280-1290.el.
9. Tripathi, K.; Feuerstein, J. D. New developments in ulcerative colitis: Latest evidence on management, treatment, and maintenance. Drugs Context 2019, 8, 212572.
10. Feuerstein, J. D.; Moss, A. C.; Farraye, F. A. Ulcerative Colitis. Mayo Clin. Proc. 2019, 94, 1357-1373.
11. Boal Carvalho, P.; Cotter, J. Mucosal Healing in Ulcerative Colitis: A Comprehensive Review. Drugs 2017, 77, 159-173.
12. Pineton de Chambrun, G.; Blanc, P.; Peyrin-Biroulet, L. Current evidence supporting mucosal healing and deep remission as important treatment goals for inflammatory bowel disease. Expert Rev. Gastroenterol. Hepatol. 2016, 10, 915-927.
13. Gunnarsson, C.; Chen, J.; Rizzo, J. A.; Ladapo, J. A.; Lofland, J. H. Direct health care insurer and out-of-pocket expenditures of inflammatory bowel disease: Evidence from a US national survey. Dig. Dis. Sci. 2012, 57, 3080-3091.
14. Leoni, G.; Neumann, P. A.; Sumagin, R.; Denning, T. L.; Nusrat, A. Wound repair: Role of immune-epithelial interactions. Mucosal Immunol. 2015, 8, 959-968.
15. Baldauf, K. J.; Royal, J. M.; Kouokam, J. C.; Haribabu, B.; Jala, V. R.; Yaddanapudi, K.; Hamorsky, K. T.; Dryden, G. W.; Matoba, N. Oral administration of a recombinant cholera toxin B subunit promotes mucosal healing in the colon. Mucosal Immunol. 2017, 10, 887-900.
16. Royal, J. M.; Reeves, M. A.; Matoba, N. Repeated Oral Administration of a KDEL-tagged Recombinant Cholera Toxin B Subunit Effectively Mitigates DSS Colitis Despite a Robust Immunogenic Response. Toxins 2019, 11, 678. [CrossRef]
17. Royal, J. M.; Matoba, N. Therapeutic Potential of Cholera Toxin B Subunit for the Treatment of Inflammatory Diseases of the Mucosa. Toxins 2017, 9, 379.
18. Hamorsky, K. T.; Kouokam, J. C.; Bennett, L. J.; Baldauf, K. J.; Kajiura, H.; Fujiyama, K.; Matoba, N. Rapid and scalable plant-based production of a cholera toxin B subunit variant to aid in mass vaccination against cholera outbreaks. PLOS Negl. Trop. Dis. 2013, 7, e2046.
19. Hamorsky, K. T.; Kouokam, J. C.; Jurkiewicz, J. M.; Nelson, B.; Moore, L. J.; Husk, A. S.; Kajiura, H.; Fujiyama, K.; Matoba, N. N-glycosylation of cholera toxin B subunit in Nicotiana benthamiana: Impacts on host stress response, production yield and vaccine potential. Sci. Rep. 2015, 5, 8003.
20. Royal, J. M.; Oh, Y. J.; Grey, M. J.; Lencer, W. I.; Ronquillo, N.; Galandiuk, S.; Matoba, N. A modified cholera toxin B subunit containing an ER retention motif enhances colon epithelial repair via an unfolded protein response. FASEB J. 2019, 33, 13527-13545.
21. Feuerstein, J. D.; Cheifetz, A. S. Ulcerative colitis: Epidemiology, diagnosis, and management. Mayo Clin. Proc. 2014, 89, 1553-1563.
22. Kim, J. J.; Shajib, M. S.; Manocha, M. M.; Khan, W. I. Investigating intestinal inflammation in DSS-induced model of IBD. J. Vis. Exp. 2012, 3678.
23. Cooper, H. S.; Murthy, S. N.; Shah, R. S.; Sedergran, D. J. Clinicopathologic study of dextran sulfate sodium experimental murine colitis. Lab. Investig. 1993, 69, 238-249.
24. Morris, D. A.; Reeves, M. A.; Royal, J. M.; Hamorsky, K. T.; Matoba, N. Isolation and detection of a KDEL-tagged recombinant cholera toxin B subunit from Nicotiana benthamiana. Process Biochem. 2021, 101, 42-49.
25. Wirtz, S.; Popp, V.; Kindermann, M.; Gerlach, K.; Weigmann, B.; Fichtner-Feigl, S.; Neurath, M. F. Chemically induced mouse models of acute and chronic intestinal inflammation. Nat. Protoc. 2017, 12, 1295-1309.
26. Nugent, S. G.; Kumar, D.; Rampton, D. S.; Evans, D. F. Intestinal luminal pH in inflammatory bowel disease: Possible determinants and implications for therapy with aminosalicylates and other drugs. Gut 2001, 48, 571-577.
27. Koziolek, M.; Grimm, M.; Becker, D.; Iordanov, V.; Zou, H.; Shimizu, J.; Wanke, C.; Garbacz, G.; Weitschies, W. Investigation of pH and Temperature Profiles in the GI Tract of Fasted Human Subjects Using the Intellicap((R)) System. J. Pharm. Sci. 2015, 104, 2855-2863.
28. Langford, A.; Bhatnagar, B.; Walters, R.; Tchessalov, S.; Ohtake, S. Drying technologies for biopharmaceutical applications: Recent developments and future direction. Dry. Technol. 2018, 36, 677-684.
29. Sollohub, K.; Cal, K. Spray drying technique: II. Current applications in pharmaceutical technology. J. Pharm. Sci. 2010, 99, 587-597.
30. Ajmera, A.; Scherlieβ, R. Stabilisation of proteins via mixtures of amino acids during spray drying. Int. J. Pharm. 2014, 463, 98-107.
31. Maltesen, M. J.; van de Weert, M. Drying methods for protein pharmaceuticals. Drug Discov. Today Technol. 2008, 5, e81-e88.
32. Liao, Y. H.; Brown, M. B.; Martin, G. P. Investigation of the stabilisation of freeze-dried lysozyme and the physical properties of the formulations. Eur. J. Pharm. Biopharm. 2004, 58, 15-24.
33. Tian, F.; Sane, S.; Rytting, J. H. calorimetric investigation of protein/amino acid interactions in the solid state. Int. J. Pharm. 2006, 310, 175-186.
34. Borde, A.; Larsson, A.; Holmgren, J.; Nygren, E. Preparation and evaluation of a freeze-dried oral killed cholera vaccine formulation. Eur. J. Pharm. Biopharm. 2011, 79, 508-518.
35. Pastor, M.; Esquisabel, A.; Marquinez, I.; Talavera, A.; Pedraz, J. L. Cellulose acetate phthalate microparticles containing Vibrio cholerae: Steps toward an oral cholera vaccine. J. Drug Target. 2014, 22, 478-487.
36. Goins, B.; Freire, E. Thermal stability and intersubunit interactions of cholera toxin in solution and in association with its cell-surface receptor ganglioside GM1. Biochemistry 1988, 27, 2046-2052.
37. Zhang, R. G.; Westbrook, M. L.; Westbrook, E. M.; Scott, D. L.; Otwinowski, Z.; Maulik, P. R.; Reed, R. A.; Shipley, G. G. The 2.4 A crystal structure of cholera toxin B subunit pentamer: Choleragenoid. J. Mol. Biol. 1995, 251, 550-562.
38. Surewicz, W. K.; Leddy, J. J.; Mantsch, H. H. Structure, stability, and receptor interaction of cholera toxin as studied by Fouriertransform infrared spectroscopy. Biochemistry 1990, 29, 8106-8111.
39. Ohrem, H. L.; Schornick, E.; Kalivoda, A.; Ognibene, R. Why is mannitol becoming more and more popular as a pharmaceutical excipient in solid dosage forms? Pharm. Dev. Technol. 2014, 19, 257-262.
40. Naini, V. Physical and Chemical Stability of Spray Dried Sugars and Protein-Sugar Molecular Mixtures for Inhalation. Ph.D. Thesis, Virginia Commonwealth University, Richmond, VA, USA, 1996.
41. Rowe, R. C.; Sheskey, P.; Quinn, M. Handbook of Pharmaceutical Excipients; Libros Digitales-Pharmaceutical Press: London, U K, 2009.
42. Maury, M.; Murphy, K.; Kumar, S.; Shi, L.; Lee, G. Effects of process variables on the powder yield of spray-dried trehalose on a laboratory spray-dryer. Eur. J. Pharm. Biopharm. 2005, 59, 565-573.
43. Jain, M.; Ganesh, L.; Manoj, B.; Randhir, C.; Shashikant, B.; Chirag, S. Spray Drying in Pharmaceutical Industry: A Review. Res. J. Pharm. Dos. Forms Technol. 2012, 4, 74-79.

The teachings of all patents, published applications and references cited herein are incorporated by reference in their entirety.

What is claimed is:

1. A method of producing an oral formulation of a cholera toxin B subunit variant, comprising:
a) mixing a phosphate-buffered saline solution of the cholera toxin B subunit variant with a saccharide excipient; and
b) spray drying the solution in an air stream.

2. The method of claim 1, wherein the cholera toxin B subunit variant comprises an endoplasmic reticulum (ER) retention sequence attached to its C-terminus.

3. The method of claim 1, wherein the cholera toxin B subunit variant comprises an amino acid sequence that has at least about 80% sequence identity to at least one sequence set forth in SEQ ID NOs: 2, 4, 6, 8, 10, 12, 14, 16, 25-29 and 34.

4. The method of claim 1, wherein the cholera toxin B subunit variant comprises an amino acid sequence set forth in SEQ ID NO:4 or 34.

5. The method of claim 1, wherein the method comprises mixing a phosphate-buffered saline solution of the cholera toxin B subunit variant with about 100 mM mannitol at about pH 7.2.

6. The method of claim 1, wherein spray drying the solution uses:
a) an inlet air temperature of about 116-122° C.;
b) an outlet air temperature of about 64-67° C.; or
c) an inlet to outlet air temperature ratio of about 1.7-2.1, or
a combination thereof.
